(19) United States
(12) Patent Application Publication
(10) Pub. No.: US 2024/0109953 A1
(43) Pub. Date: Apr. 4, 2024

(54) APOLIPOPROTEIN B (APOB) POLYPEPTIDES FOR BLOCKING ENDOTHELIAL CELL UPTAKE OF APOLIPOPROTEIN B-CONTAINING LIPOPROTEINS

(71) Applicant: New York University, New York, NY (US)

(72) Inventors: Ira J. Goldberg, New York, NY (US); Ainara González Cabodevilla, Brooklyn, NY (US); M. Mahmood Hussain, Woodbury, NY (US)

(73) Assignee: New York University, New York, NY (US)

(21) Appl. No.: 18/195,473

(22) Filed: May 10, 2023

Related U.S. Application Data
(60) Provisional application No. 63/340,299, filed on May 10, 2022.

(51) Int. Cl. C07K 14/775; A61P 9/10

(52) U.S. Cl. CPC C07K 14/775; A61P 9/10; A61K 38/00

(57) ABSTRACT

The present disclosure provides polypeptides comprising an N-terminal portion(s) of Apolipoprotein B100 (ApoB100), or functional derivatives thereof, particularly polypeptides may be capable of, e.g., inhibiting scavenger receptor-B1 (SR-B1) and/or activin receptor-like kinase 1 (ALK1). Further provided are related polynucleotides, vectors, and pharmaceutical compositions. Methods for blocking endothelial cell uptake and/or transcytosis of lipoproteins comprising chylomicrons, low-density lipoprotein (LDL), very-low-density lipoprotein (VLDL), and/or lipoprotein (a) (Lp(a)) and treating atherosclerosis using the polypeptides and/or pharmaceutical compositions are also provided.

### CROSS-REFERENCE TO RELATED APPLICATIONS

This application claims the benefit of Provisional U.S. Application No. 63/340,299, filed May 10, 2022, the contents of which are incorporated by reference in their entireties for all purposes.

### STATEMENT REGARDING FEDERALLY SPONSORED RESEARCH

This invention was made with government support under HL092969 and HL45095 awarded by National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on May 23, 2023, is named 243735_000279_SL.xml and is 14,309 bytes in size.

### FIELD OF THE INVENTION

The present disclosure provides polypeptides derived from Apolipoprotein B (ApoB), or functional derivatives thereof. Further provided are related polynucleotides, vectors, and pharmaceutical compositions. Methods for blocking endothelial cell uptake and/or transcytosis of Apolipoprotein B-containing lipoproteins, such as chylomicrons, low-density lipoprotein (LDL), very-low-density lipoprotein (VLDL) and/or lipoprotein (a) (Lp(a)), and for treating atherosclerosis using the polypeptides and/or pharmaceutical compositions are also provided.

### BACKGROUND

The development of atherosclerosis, the cause of most heart disease, is initiated by the infiltration of low-density lipoprotein (LDL) into the arterial wall and subsequent recruitment of immune-inflammatory cells. This process begins with the movement of LDL into and then across endothelial cells, which line the arteries. In particular, apolipoprotein B (ApoB)-containing LDL and chylomicron remnants cross endothelial cells (ECs) and are then retained within the subendothelial layer. LDL has been reported to cross the endothelial cell (EC) barrier via two receptors: scavenger receptor-B1 (SR-B1) and activin receptor-like kinase 1 (ALK1). Lipoprotein (a) (Lp(a)) is comprised of apolipoprotein (a) (apo(a)) that associates with LDL and is another atherogenic lipoprotein that, like LDL and chylomicrons, contains ApoB which can bind to ALK1 and SR-B1. Lp(a) is an additional risk factor for coronary artery disease and atherosclerosis, and is the primary genetic association with aortic valve stenosis.

### SUMMARY OF THE INVENTION

As specified in the Background section above, there is a great need in the art for methods and compositions relating to inhibition of ApoB uptake and/or transcytosis by endothelial cells and, in particular, methods for treating atherosclerosis and/or an associated disease, disorder, or condition thereof using such compositions. The present application addresses these and other needs.

In one aspect, provided herein is an isolated polypeptide which comprises an N-terminal portion of Apolipoprotein B100 (ApoB100), or a functional derivative thereof, wherein said polypeptide does not comprise any ApoB100 sequences beyond amino acid residue 816 of ApoB100.

In some embodiments, the ApoB100 has the amino acid sequence of SEQ ID NO: 1.

In some embodiments, the polypeptide is capable of inhibiting scavenger receptor-B1 (SR-B1) and/or activin receptor-like kinase 1 (ALK1).

In some embodiments, the polypeptide does not comprise amino acid residue 816 of ApoB100.

In some embodiments, the polypeptide comprises amino acid residues 363-544 of ApoB100, or a derivative thereof. In some embodiments, the polypeptide comprises the amino acid sequence of SEQ ID NO: 5, or a derivative thereof. In some embodiments, the polypeptide consists of the amino acid sequence of SEQ ID NO: 5, or a derivative thereof.

In some embodiments, the polypeptide comprises amino acid residues 1-544 of ApoB100, or a derivative thereof. In some embodiments, the polypeptide comprises the amino acid sequence of SEQ ID NO: 4, or a derivative thereof. In some embodiments, the polypeptide consists of the amino acid sequence of SEQ ID NO: 4, or a derivative thereof.

In some embodiments, the polypeptide comprises amino acid residues 544-684 of ApoB100, or a derivative thereof. In some embodiments, the polypeptide comprises the amino acid sequence of SEQ ID NO: 7, or a derivative thereof. In some embodiments, the polypeptide consists of the amino acid sequence of SEQ ID NO: 7, or a derivative thereof.

In some embodiments, the polypeptide comprises amino acid residues 363-684 of ApoB100, or a derivative thereof. In some embodiments, the polypeptide comprises the amino acid sequence of SEQ ID NO: 6, or a derivative thereof. In some embodiments, the polypeptide consists of the amino acid sequence of SEQ ID NO: 6, or a derivative thereof.

In some embodiments, the polypeptide comprises amino acid residues 1-684 of ApoB100, or a derivative thereof. In some embodiments, the polypeptide comprises the amino acid sequence of SEQ ID NO: 3, or a derivative thereof. In some embodiments, the polypeptide consists of the amino acid sequence of SEQ ID NO: 3, or a derivative thereof.

In another aspect, provided herein is a polynucleotide comprising a nucleotide sequence encoding the polypeptide of any one of the embodiments described herein. In some embodiments, the polynucleotide is RNA. In some embodiments, the RNA is mRNA. In some embodiments, the polynucleotide is DNA. In some embodiments, the polynucleotide is incorporated into liposomes.

In another aspect, provided herein is a recombinant vector comprising the polynucleotide of any one of the embodiments described herein.

In some embodiments, the vector is a viral vector. In some embodiments, the viral vector has a tropism for liver. In some embodiments, the viral vector is an adeno-associated virus (AAV) vector, an adenovirus, or a lentivirus. In some embodiments, the AAV vector is AAV8 vector or AAV2 vector.

In some embodiments, the vector is a non-viral vector. In some embodiments, the vector is a plasmid.

In various embodiments, the polynucleotide is operably linked to one or more regulatory sequences (e.g., a promoter) for expression of the polypeptide.

In another aspect, provided herein is a pharmaceutical composition comprising (i) the polypeptide of any one of the embodiments described herein, (ii) the polynucleotide of any one of the embodiments described herein, or (iii) the recombinant vector of any one of the embodiments described herein, and a pharmaceutically acceptable carrier or adjuvant.

In a further aspect, provided herein is a method of blocking uptake or transcytosis of low-density lipoprotein (LDL), very-low-density-lipoprotein (VLDL), chylomicrons, and/or lipoprotein (a) (Lp(a)) by an endothelial cell, comprising contacting the endothelial cell with an effective amount of an inhibitor of scavenger receptor-B1 (SR-B1) and/or activin receptor-like kinase 1 (ALK1). In some embodiments, the endothelial cell is an aortic endothelial cell. In some embodiments, the cell is in a subject and the inhibitor of SR-B1 and/or ALK1 is administered to the subject. In some embodiments, the subject has atherosclerosis or an associated disease, disorder, or condition thereof.

In a further aspect, provided herein is a method of treating or preventing atherosclerosis or an associated disease, disorder, or condition thereof in a subject in need thereof, comprising administering to the subject a therapeutically effective amount of an inhibitor of scavenger receptor-B1 (SR-B1) and/or activin receptor-like kinase 1 (ALK1). In some embodiments, the disease, disorder, or condition associated with is atherosclerosis is an inflammatory disease, obesity, metabolic disease, or eruptive xanthomas.

In various embodiments, the inhibitor inhibits the interaction between ApoB100 with SR-B1 and/or ALK1.

In various embodiments, the inhibitor of SR-B1 and/or ALK1 comprises (i) the polypeptide of any one of the embodiments described herein; (ii) the polynucleotide of any one of the embodiments described herein; (iii) the recombinant vector of any one of the embodiments described herein; or (iv) the pharmaceutical composition described herein.

In some embodiments, the inhibitor of SR-B1 and/or ALK1 comprises a polypeptide consisting of amino acid residues 1-816 of ApoB100, or a derivative thereof; a polynucleotide encoding said polypeptide or derivative thereof, a recombinant vector comprising said polynucleotide; or a pharmaceutical composition comprising said polypeptide or derivative thereof, said polynucleotide, or said recombinant vector. In some embodiments, the polypeptide consists of SEQ ID NO: 2, or a derivative thereof.

In some embodiments, the inhibitor of SR-B1 and/or ALK1 is a small molecule (e.g., a synthetic compound or a plant derived compound), an antibody, an siRNA, an shRNA, or an antisense oligonucleotide.

In various embodiments, the inhibitor is administered intravenously, subcutaneously, orally, or intranasally.

In various embodiments, the inhibitor is administered with an additional agent. In some embodiments, the additional agent is semaglutide.

In various embodiments, the subject is human.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows an exemplary ApoB amino acid sequence (SEQ ID NO: 2).

FIGS. 2A-2D depict a comparison of N-terminal ApoB fragment inhibition of chylomicron and low-density lipoprotein (LDL) uptake by endothelial cells (ECs). Detection of flag-tagged carboxyl-truncated ApoB polypeptides (e.g., ApoB3, ApoB5, ApoB15, and ApoB18) via agarose gel electrophoresis (FIG. 2A). Mouse endothelial cells (ECs) were deprived of serum overnight, then incubated for 30 minutes with 1,1′-Dioctadecyl 3,3,3′,3′-Tetramethylindocarbocyanine Perchlorate (DiI; red fluorescent)-labeled chylomicrons (4 mg/ml triglyceride [TG]) or LDL (2.5 mg/dl) in the absence (control) or presence of 0.4 mg/dL ApoB polypeptides. ApoB15 and ApoB18 block endothelial cell chylomicron uptake (FIG. 2B). The inhibition requires a fragment that is longer than ApoB12. ApoB12 blocks EC DiI-LDL uptake (FIGS. 2C-2D). Thus, the ALK1 binding site is between ApoB8 and ApoB12.

FIGS. 3A-3B show ApoB18 expressing adeno-associated virus (AAV) reduces lipid droplet accumulation in lipoprotein lipase (LpL) deficient hypertriglyceridemic mice. Lpl^{fl/fl} (floxed control) and hypertriglyceridemic iLpl^{-/-} mice were injected with either control (null AAV) or ApoB18 AAV (2.00E+11 vg/mouse). Blood was obtained 8 weeks after the injection and run on a polyacrylamide gel (10%) (FIG. 3A, top panel). ApoB and its fragments were identified by western blot. Plasma of mice treated with ApoB18 AAV contained high levels of circulating ApoB18. The western blot shows expression of the ApoB18 peptide at levels at least equal to that of full-length ApoB48 in LpL deficient mice. Thus, levels of ApoB18 were similar to those of circulating ApoB48 and ApoB100. These data demonstrate that ApoB18 is expressed in mice after AAV injection (FIG. 3A, bottom panel). Mice were sacrificed 180 minutes after an olive oil gavage and their aortas were stained with EC marker vascular endothelial (VE)-cadherin (red) and BODIPY (green) to assess postprandial lipid droplet accumulation, a marker for chylomicron uptake. Mice treated with ApoB18 AAV exhibited significantly fewer postprandial aortic EC lipid droplets (FIG. 3B). Thus, ApoB18 expression reduced lipid droplet accumulation in aortic ECs. AAV18 did not alter circulating lipoprotein levels in these mice.

FIGS. 4A-4E show treatment with ApoB18 AAV reduces atherosclerosis in low-density lipoprotein receptor (LDLR) knock out (KO) mice. For the experimental design, LDLR KO mice were injected at 8 weeks of age with 2.00E+11 vg/mouse null AAV (control virus) or with AAV expressing ApoB 18 (ApoB18 AAV). Mice were fed a western diet (WD), enriched in cholesterol and fat, for 12 weeks to induce atherosclerosis. After 12 weeks, mice were sacrificed and their aortic roots were preserved and sectioned to assess atherosclerotic lesion size and lipid content (FIG. 4A). Aortic roots were stained with hematoxylin/eosin for evaluation of atherosclerotic lesions (i.e., aortic root lesions) and then with Oil Red O (ORO) to stain the neutral lipid (cholesterol) (FIG. 4B, top panel). Mice treated with ApoB18 AAV exhibited significantly smaller aortic root lesions (FIG. 4B, bottom panel, left). The percent of the aortic root lesion which was occupied by the lipid was similar between the two groups, with a trend towards reduction of lipid content in mice treated with ApoB18 AAV, i.e., the lesions were smaller in mice treated with ApoB18 AAV but similar in size to those in the control (null AAV) group, as assessed by Oil Red O staining (FIG. 4B, bottom panel, right). There was no significant difference in weight gain (g) (FIG. 4C), plasma cholesterol (TC, total cholesterol) (mg/dL) (FIG. 4D), or plasma triglyceride (TG) (mg/dL TG) (FIG. 4E) levels between mice treated with null AAV versus ApoB18 AAV. Data depicted in FIGS. 4A-4E illustrate that ApoB18 AAV significantly reduced aortic lesion area after 12 weeks of western diet (WD) but did not profoundly alter percent lesion lipid content as monitored by ORO staining, and that plasma cholesterol, triglyceride, and weight was identical regardless of treatment after WD and at time of sacrifice.

FIGS. 5A-5B show activin receptor-like kinase 1 (ALK1) small interfering RNA (siRNA) and ApoB12 peptide inhibit endothelial cell (EC) uptake of ApoB100-carrying chylomicrons (CMs). Endothelial cells were deprived of serum overnight and exposed to wild type (WT; ApoB48-carrying) or ApoBec1^{-/-} (ApoB100-carrying) chylomicrons (4 mg/dL TG) for 30 minutes. There was no significant difference in ApoB48-carrying (B48) versus ApoB100-carrying (B100) CM uptake by ECs (FIG. 5A). Inhibition of ALK1 with siRNA and co-incubation with ApoB12 peptide inhibited EC uptake of ApoBec1^{-/-} chylomicrons but not WT chylomicrons (FIG. 5B).

FIGS. 6A-6B show treatment with chylomicrons induces transcription of proinflammatory genes in endothelial cells. Endothelial cells (ECs) were deprived of serum overnight and either left untreated (control) or exposed to chylomicrons (4 mg/dL TG) for 30 minutes. Cells were then thoroughly washed with phosphate buffered saline (PBS) to remove non-internalized chylomicrons, and maintained for 16 hours in fetal bovine serum (FBS)-free culture medium in the presence or absence (+/−) of lysosome proton pump inhibitor Bafilomycin A1 (BafA1) to inhibit intracellular chylomicron hydrolysis. RNA-sequencing (RNA-seq) data showing enhanced expression of proinflammatory genes. The top 10 transcripts induced by chylomicron treatment were inflammatory markers (FIG. 6A). A heatmap displaying transcriptome profile categorization for the inflammatory genes from the RNA-Seq dataset (FIG. 6B).

FIGS. 7A-7E show the amino acid sequences of the exemplary ApoB polypeptides disclosed herein.

FIGS. 8A-8B demonstrate that ApoB18 AAV reduces atherosclerotic lesion area in the aortic arch. Images of the aortic arch of mice injected with null AAV compared to ApoB18 AAV show atherosclerotic plaques (white) (FIG. 8A). The images were analyzed by quantifying the percent of the aortic arch covered with white plaque (FIG. 8B). The data show that the percent lesion area was reduced in the ApoB18 AAV treatment group as compared to the control group (null AAV).

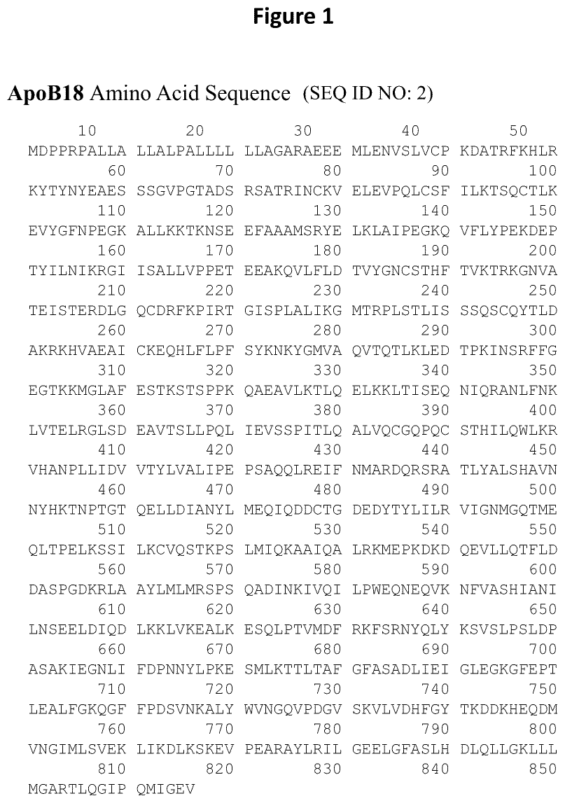

### DETAILED DESCRIPTION

The development of atherosclerosis, the cause of most heart disease, is initiated by the infiltration of low-density lipoprotein (LDL) into the arterial wall and subsequent recruitment of immune-inflammatory cells. This process begins with the movement of LDL into and then across endothelial cells, which line the arteries. While not wishing to be bound by theory, two receptors, scavenger receptor B1 (SR-B1) and activin receptor-like kinase (ALK1) are believed to mediate this process. A fragment containing 18% of the N-terminal region of the major LDL protein, ApoB, blocks SR-B1-mediated uptake of chylomicrons by endothelial cells. This peptide, ApoB18 (SEQ ID NO: 2), also blocks uptake of LDL by endothelial cells, likely because it inhibits LDL interaction with both receptors, SR-B1 and ALK1.

Studies of the present disclosure define the regions of ApoB required for binding to SR-B1 and ALK1. Shorter ApoB polypeptides (non-lipoprotein ApoB peptides) block chylomicron and/or LDL uptake by endothelial cells. Scientific findings described herein show the region required to block LDL uptake, and ALK1, requires an N-terminal ApoB fragment containing at least ApoB12 (SEQ ID NO: 4); inhibition of chylomicron uptake requires a longer fragment, at least ApoB15 (SEQ ID NO: 3). Thus, the two receptors likely interact with different regions of ApoB.

The present disclosure also describes, among other things, recombinant vectors such as adeno-associated virus (AAV) encoding ApoB polypeptides of the present disclosure. For example, an AAV encoding ApoB18 was designed to test the anti-atherogenic effects of ApoB18 in vivo. Plasma from mice treated with ApoB18 AAV contained high levels of circulating ApoB18. ApoB18 AAV reduced the accumulation of chylomicron-derived postprandial lipid droplets in mouse aortas, evidence that it blocks SR-B1 in vivo. Whether ApoB18 would affect atherosclerosis was also tested using hypercholesterolemic LDL receptor (LDLR) knock out mice treated with either control (null AAV) or ApoB18 AAV and fed a western diet to induce atherosclerosis. Mice were sacrificed and their aortic roots were harvested for assessment of atherosclerotic lesions. Mice treated with ApoB18 AAV exhibited markedly smaller lesions, and reduced lesion lipid content.

Examples of the present disclosure reveal that shorter ApoB polypeptides and other small molecules can inhibit the binding of LDL to SR-B1. In some embodiments, an ApoB100 fragment is created that spans the ligand binding site of both receptors. The precise regions of receptor interaction for generation of small polypeptides and/or small molecules to block both receptors is defined.

Whether the ApoB100 exposes a binding site between ApoB8 and ApoB12 that is masked in ApoB48 is tested via obtaining blood after a fat meal from mice that only express ApoB100, and ApoB100 chylomicron uptake was blocked by ALK1 knockdown. How chylomicron uptake affects endothelial gene expression was assessed using RNA-seq analysis, and the top transcripts induced by this treatment were inflammatory markers. Conventional markers associated with greater atherosclerosis were also upregulated. Blocking endothelial cell uptake by aortic endothelial cells using methods and compositions disclosed herein is anti-inflammatory, and prevents or reduces atherosclerosis.

### Definitions

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures used in connection with, and techniques of, cell and tissue culture, molecular biology, immunology, microbiology, genetics and protein and nucleic acid chemistry and hybridization described herein are those well-known and commonly used in the art.

The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2d ed., Cold Spring Harbor Laboratory Press, Cold Spring Harbor, N.Y. (1989) and Ausubel et al., Current Protocols in Molecular Biology, Greene Publishing Associates (1992), and Harlow and Lane Antibodies: A Laboratory Manual, Cold Spring Harbor Laboratory Press, Cold Spring Harbor, N.Y. (1990), which are incorporated herein by reference. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The following terms, unless otherwise indicated, shall be understood to have the following meanings:

The terms “polypeptide” and “protein” used interchangeably herein encompass native or artificial proteins, protein fragments and polypeptide analogs of a protein sequence. A polypeptide or protein may be monomeric or polymeric.

The term “fragment” in regard to polypeptides refers to a polypeptide that has an amino-terminal, carboxy-terminal deletion and/or internal deletion, but where the remaining amino acid sequence is substantially identical to the corresponding positions in the full-length naturally-occurring sequence. Also, fragments according to the invention may be made by truncation, e.g., by removal of one or more amino acids from the N and/or C-terminal ends of a polypeptide. Up to 10, up to 50, up to 100, up to 200, up to 400, up to 600, up to 800, up to 1000, up to 1500, up to 2000, up to 2500, up to 3000, up to 3500 or more amino acids may be removed from the N and/or C terminal in this way. Fragments may also be generated by one or more internal deletions. In some embodiments, fragments are at least 50, 100, 150, 200, 250, 300, 350, 400, 450, 500, 550, 600, 650, 700, 750, or 800 amino acids long. In some embodiments, a fragment may comprise an amino acid sequence of at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 amino contiguous acid residues, at least 200 contiguous amino acid residues, at least contiguous 250 amino acid residues, at least 300 contiguous amino acid residues, at least contiguous 350 amino acid residues, at least 400 contiguous amino acid residues, at least contiguous 450 amino acid residues, at least 500 contiguous amino acid residues, at least contiguous 550 amino acid residues, at least 600 contiguous amino acid residues, at least contiguous 650 amino acid residues, at least 700 contiguous amino acid residues, at least contiguous 750 amino acid residues, at least 800 contiguous amino acid residues, at least 850 contiguous amino acid residues of the amino acid sequence of the full-length polypeptide or protein. In some embodiments, the fragment may comprise 141 contiguous amino acid residues of the full-length polypeptide or protein. In some embodiments, the fragment may comprise 182 contiguous amino acid residues of the full-length polypeptide or protein. In some embodiments, the fragment may comprise 322 contiguous amino acid residues of the full-length polypeptide or protein. In some embodiments, the fragment may comprise 544 contiguous amino acid residues of the full-length polypeptide or protein. In some embodiments, the fragment may comprise 684 contiguous amino acid residues of the full-length polypeptide or protein. In some embodiments, the fragment may comprise 816 contiguous amino acid residues of the full-length polypeptide or protein.

The term “derivative” as used herein refers to a polypeptide, or a variant, or an analog thereof, comprising one or more alterations and/or chemical modifications as compared to a corresponding reference wild-type polypeptide (e.g., ApoB100 or fragments thereof). In some embodiments, the term “derivative” may refer to, for example: (a) a polypeptide that has at least 40%, 45%, 50%, 55%, 60%, 65%, 70%, 75%, 80%, 85%, 90%, 95%, 98% or 99% sequence identity to the polypeptide it is a derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a derivative of; (c) a polypeptide that contains 1, 2, 3, 4, 5, 6, 7, 8, 9, 10, 11, 12, 13, 14, 15, 16, 17, 18, 19, 20 or more amino acid mutations (e.g., additions, deletions and/or substitutions) relative to the polypeptide it is a derivative of; (d) a polypeptide encoded by nucleic acids can hybridize under high, moderate or typical stringency hybridization conditions to nucleic acids encoding the polypeptide it is a derivative of; (e) a polypeptide encoded by a nucleotide sequence that can hybridize under high, moderate or typical stringency hybridization conditions to a nucleotide sequence encoding a fragment of the polypeptide, it is a derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a derivative of (e.g., N-terminal fragment).

For a detailed description of protein chemistry and structure, see Schulz, G. E. et al., PRINCIPLES OF PROTEIN STRUCTURE, Springer-Verlag, New York, 1978, and Creighton, T. E., PROTEINS: STRUCTURE AND MOLECULAR PROPERTIES, W. H. Freeman & Co., San Francisco, 1983, which are hereby incorporated by reference. The types of substitutions which may be made in the polypeptides of the present invention may be based on analysis of the frequencies of amino acid changes between homologous polypeptides of different species. Based on such an analysis, conservative substitutions are defined herein as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gln;
3. Polar, positively charged residues: His, Arg, Lys;
4. Large aliphatic, nonpolar residues: Met, Leu, Ile, Val (Cys); and
5. Large aromatic residues: Phe, Tyr, Trp.

The three amino acid residues in parentheses above have special roles in protein architecture. Gly is the only residue lacking any side chain and thus imparts flexibility to the chain. Pro, because of its unusual geometry, tightly constrains the chain. Cys can participate in disulfide bond formation which is important in protein folding. Note the Schulz et al. would merge Groups 1 and 2, above. Note also that Tyr, because of its hydrogen bonding potential, has some kinship with Ser, Thr, etc.

Preferred deletions, insertions, and substitutions, according to the present invention, are those which do not produce radical changes in the characteristics of the polypeptide molecule. However, when it is difficult to predict the exact effect of the substitution, deletion, or insertion in advance of doing so, one skilled in the art will appreciate that the effect will be evaluated by routine screening assays which are described in more detail below. For example, a change in the immunological character of a polypeptide, such as binding to a given receptor, is measured by a competitive type immunoassay. Biological activity is screened in an appropriate bioassay, as described below.

Modifications of such polypeptide properties as redox or thermal stability, hydrophobicity, susceptibility to proteolytic degradation or the tendency to aggregate with carriers or into multimers can be assayed by methods well known to the ordinarily skilled artisan.

Derivatives of ApoB polypeptides described herein can be prepared by direct chemical synthesis or by genetic recombinant technology, using methods well-known in the art. For example, the polypeptides of the invention can be synthesized substantially free of other proteins or glycoproteins of mammalian origin in a prokaryotic organism, in a non-mammalian eukaryotic organism, by a yeast, or by a baculovirus system, if desired. Alternatively, methods are well known for the synthesis of polypeptides of desired sequence on solid phase supports and their subsequent separation from the support. Derivatives of ApoB polypeptides described herein comprising mutations can be prepared by site-directed mutagenesis (as exemplified by Adelman et al., DNA 2:183 (1983)) of nucleotides in the DNA encoding said polypeptides, and thereafter expressing the DNA (cDNA, RNA, and protein) in recombinant cell culture.

Derivatives of ApoB polypeptides encompassed by the present invention include “chemical derivatives” containing additional chemical moieties not normally a part of a polypeptide. Covalent modifications of the polypeptides are included within the scope of this invention. Such modifications may be introduced into the molecule by reacting targeted amino acid residues of the polypeptide with an organic derivatizing agent that is capable of reacting with selected side chains or terminal residues.

Additionally, modified amino acids or chemical derivatives of amino acids of ApoB polypeptides, according to the present invention may be provided, which polypeptides contain additional chemical moieties or modified amino acids not normally a part of the protein. Covalent modifications of the polypeptide are thus included within the scope of the present invention. The following examples of chemical derivatives are provided by way of illustration and not by way of limitation.

Aromatic amino acids may be replaced with D- or L-naphthylalanine, D- or L-phenylglycine, D- or L-2-thienylalanine, D- or L-1-, 2-, 3- or 4-pyrenylalanine, D- or L-3-thienylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylphenylalanine, D- or L-p-methoxybiphenylphenylalanine, D- or L-2-indole(alkyl)alanine, and D- or L-alkylalanine where alkyl may be substituted or unsubstituted methyl, ethyl, propyl, hexyl, butyl, pentyl, isopropyl, isobutyl, sec-isotyl, isopentyl, non-acidic amino acids, of chain lengths of C1-C20.

Acidic amino acids can be substituted with non-carboxylate amino acids while maintaining a negative charge, and derivatives or analogs thereof, such as the non-limiting examples of (phosphono)-alanine, glycine, leucine, isoleucine, threonine, or serine; or sulfated (for example, —SO₃H) threonine, serine, tyrosine.

Other substitutions may include unnatural hydroxylated amino acids may made by combining “alkyl” with any natural amino acid. Basic amino acids may be substituted with alkyl groups at any position of the naturally occurring amino acids lysine, arginine, ornithine, citrulline, or (guanidino)-acetic acid, or other (guanidino)alkyl-acetic acids. Nitrile derivatives (for example, containing the CN-moiety in place of COOH) may also be substituted for asparagine or glutamine, and methionine sulfoxide may be substituted for methionine. Methods of preparation of such peptide derivatives are well known to one skilled in the art.

In addition, any amide linkage the polypeptides can be replaced by a ketomethylene moiety. Such derivatives are expected to have the property of increased stability to degradation by enzymes, and therefore possess advantages for the formulation of compounds which may have increased in vivo half-lives, as administered by various routes as described herein.

In addition, any amino acid representing a component of the peptides can be replaced by the same amino acid but of the opposite chirality. Thus, any amino acid naturally occurring in the L-configuration (which may also be referred to as the R or S, depending upon the structure of the chemical entity) may be replaced with an amino acid of the same chemical structural type, but of the opposite chirality, generally referred to as the D-amino acid but which can additionally be referred to as the R- or the S-, depending upon its composition and chemical configuration. Such derivatives have the property of greatly increased stability to degradation by enzymes, and therefore are advantageous in the formulation of compounds which may have longer in vivo half-lives, when administered by various routes.

Additional amino acid modifications in the ApoB polypeptides described herein may include the following.

Cysteinyl residues most commonly are reacted with a-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, a-bromo-13-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues are derivatized by reaction with diethylprocarbonate at pH 5.5-7.0 because this agent is relatively specific for the histidyl side chain. Para-bromophenacyl bromide also is useful; the reaction is preferably performed in 0.1M sodium cacodylate at pH 6.0.

Lysinyl and amino terminal residues are reacted with succinic or other carboxylic acid anhydrides, which reverses the charge of the lysinyl residues. Other suitable reagents for derivatizing a-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine c-amino group.

The specific modification of tyrosyl residues has been studied extensively with particular interest in introducing spectral labels into tyrosyl residues by reaction with aromatic diazonium compounds or tetranitromethane. Most commonly, N-acetylimidizol and tetranitromethane are used to form 0-acetyl tyrosyl species and 3-nitro derivatives, respectively.

Carboxyl side groups (aspartyl or glutamyl) are selectively modified by reaction with carbodiimides (R′—N—C—N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Glutaminyl and asparaginyl residues are deamidated to the corresponding glutamyl and aspartyl residues. Alternatively, these residues are deamidated under mildly acidic conditions. Either form of these residues falls within the scope of this invention.

Derivatization with bifunctional agents is useful for cross-linking the peptide to a water-insoluble support matrix or to other macromolecular carriers. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis-(succinimidyl-propionate), and bifunctional maleimides such as bis-N-maleimido-1,8-octane. Derivatizing agents such as methyl-3-[(p-azidophenyl)dithic]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Other modifications include hydroxylation of proline and lysine, phosphorylation of hydroxyl groups of seryl or threonyl residues, methylation of the a-amino groups of lysine, arginine, and histidine side chains (Creighton, supra), acetylation of the N-terminal amine, and, in some instances, amidation of the C-terminal carboxyl groups.

Such derivatized moieties may improve the solubility, absorption, biological half-life, and the like. The moieties may alternatively eliminate or attenuate any undesirable side effect of the polypeptide and the like. Moieties capable of mediating such effects are disclosed, for example, in Remington's Pharmaceutical Sciences, 16th ed., Mack Publishing Co., Easton, Pa. (1980).

The term “functional derivative” as used herein refers to a derivative of the polypeptide or protein, or a polynucleotide encoding the polypeptide or protein that retains at least one function of the full-length polypeptide or protein. A functional derivative may comprise one, two, three, or more fragments of the full-length polypeptide or protein, or polynucleotide encoding the polypeptide or protein. For example, a functional derivative of an ApoB polypeptide described herein may be a polypeptide derivative sufficient for inhibiting scavenger receptor-B1 (SR-B1) and/or activin receptor-like kinase 1 (ALK1).

In certain embodiments, amino acid substitutions of a protein or portion thereof are those which: (1) reduce susceptibility to proteolysis, (2) reduce susceptibility to oxidation, (3) alter binding affinity for forming protein complexes, or (4) confer or modify other physicochemical or functional properties. For example, single or multiple amino acid substitutions (preferably conservative amino acid substitutions) may be made in the normally-occurring sequence.

A conservative amino acid substitution should not substantially change the structural characteristics of the parent sequence. Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al., Nature 354:105 (1991), which are each incorporated herein by reference.

As used herein, the twenty naturally occurring amino acids and their abbreviations follow conventional usage. See Immunology—A Synthesis (2nd Edition, E. S. Golub and D. R. Gren, Eds., Sinauer Associates, Sunderland, Mass. (1991)), which is incorporated herein by reference.

The term “polynucleotide” as referred to herein means a polymeric form of nucleotides of at least 10 bases in length, either ribonucleotides or deoxyribonucleotides or a modified form of either type of nucleotide. The term includes single and double stranded forms.

The term “oligonucleotide” as used herein includes naturally occurring, and modified nucleotides linked together by naturally occurring and non-naturally occurring oligonucleotide linkages. Oligonucleotides are a polynucleotide subset generally comprising a length of 200 bases or fewer. Preferably oligonucleotides are 10 to 60 bases in length and in some embodiments 12, 13, 14, 15, 16, 17, 18, 19, or 20 to 40 bases in length. Oligonucleotides are usually single stranded, e.g. for primers and probes; although oligonucleotides may be double stranded, e.g. for use in the construction of a gene mutant. Oligonucleotides of the invention can be either sense or antisense oligonucleotides.

The term “antibody” as used herein refers to an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Allazikani et al (1997) J. Molec. Biol. 273:927-948; and Almagro, J. Mol. Recognit. 17:132-143 (2004). See also hgmp.mrc.ac.uk and bioinf.org.uk/abs).

An antibody described herein may be a full-length antibody, which contains two heavy chains and two light chains, each including a variable domain and a constant domain. Alternatively, an antibody can be an antigen-binding fragment of a full-length antibody. Examples of binding fragments encompassed within the term “antigen-binding fragment” of a full length antibody include (i) a Fab fragment, a monovalent fragment consisting of the VL, VH, CL and CHi domains; (ii) a F(ab′)2 fragment, a bivalent fragment including two Fab fragments linked by a disulfide bridge at the hinge region; (iii) a Fd fragment consisting of the VH and CHi domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

A “small molecule” is a compound having molecular mass of less than 3000 Daltons, preferably less than 2000 or 1500, still more preferably less than 1000, and most preferably less than 600 Daltons. In some embodiments, the small molecule described herein is a synthetic compound or a plant derived compound.

“Operably linked” sequences include both expression control sequences that are contiguous with the gene of interest and expression control sequences that act in trans or at a distance to control the gene of interest. The term “expression control sequence” as used herein means polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

The term “vector”, as used herein, means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional DNA segments may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”).

The term “promoter” as used herein is defined as a DNA sequence recognized by the synthetic machinery of the cell, or introduced synthetic machinery, required to initiate the specific transcription of a polynucleotide sequence. As used herein, the term “regulatory sequence” means a nucleic acid sequence which can regulate expression of a gene product operably linked to the regulatory sequence. In some instances, this sequence may be the core promoter sequence and in other instances, this sequence may also include an enhancer sequence and other regulatory elements which are required for expression of the gene product. The promoter or regulatory sequence may, for example, be one which expresses the gene product in a tissue specific manner. The term “percent sequence identity” means a ratio, expressed as a percent of the number of identical residues over the number of residues compared.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

Sequence identity for nucleic acid sequences may be analyzed over a stretch of at least about nine nucleotides, usually at least about 18 nucleotides, more usually at least about 24 nucleotides, typically at least about 28 nucleotides, more typically at least about 32 nucleotides, and preferably at least about 36, 48 or more nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA, which includes, e.g., the programs FASTA2 and FASTA3, provides alignments and percent sequence identity of the regions of the best overlap between the query and search sequences (Pearson, Methods Enzymol. 183:63-98 (1990); Pearson, Methods Mol. Biol. 132:185-219 (2000); Pearson, Methods Enzymol. 266:227-258 (1996); Pearson, J. Mol. Biol. 276:71-84 (1998); herein incorporated by reference). Unless otherwise specified, default parameters for a particular program or algorithm are used. For instance, percent sequence identity between nucleic acid sequences can be determined using FASTA with its default parameters (a word size of 6 and the NOPAM factor for the scoring matrix) or using Gap with its default parameters as provided in GCG Version 6.1, herein incorporated by reference.

A reference to a nucleotide sequence encompasses its complement unless otherwise specified. Thus, a reference to a nucleic acid having a particular sequence should be understood to encompass its complementary strand, with its complementary sequence.

Sequence identity for polypeptides, is typically measured using sequence analysis software. Protein analysis software matches sequences using measures of similarity assigned to various substitutions, deletions and other modifications, including conservative amino acid substitutions. For instance, GCG contains programs such as “Gap” and “Bestfit” which can be used with default parameters, as specified with the programs, to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA using default or recommended parameters, see GCG Version 6.1. (University of Wisconsin Wis.) FASTA (e.g., FASTA2 and FASTA3) provides alignments and percent sequence identity of the regions of the best overlap between the query and search sequences (Pearson, Methods Enzymol. 183:63-98 (1990); Pearson, Methods Mol. Biol. 132:185-219 (2000)). Another preferred algorithm when comparing a sequence of the invention to a database containing a large number of sequences from different organisms is the computer program BLAST, especially blastp or tblastn, using default parameters, as supplied with the programs. See, e.g., Altschul et al., J. Mol. Biol. 215:403-410 (1990); Altschul et al., Nucleic Acids Res. 25:3389-402 (1997).

The length of polypeptide sequences compared for homology will generally be at least about 16 amino acid residues, usually at least about 20 residues, more usually at least about 24 residues, typically at least about 28 residues, and preferably more than about 35 residues. When searching a database containing sequences from a large number of different organisms, it is preferable to compare amino acid sequences.

The terms “inhibit” or “inhibition” as used herein refer to reducing a function or activity to an extent sufficient to achieve a desired biological or physiological effect. Inhibition may be complete or partial.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

As used herein, “pharmaceutically acceptable carrier” or “pharmaceutical acceptable excipient” includes any material which, when combined with an active ingredient, allows the ingredient to retain biological activity and is non-reactive with the subject's immune system. Compositions comprising such carriers are formulated by well-known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject. For the avoidance of doubt, reference herein to “treatment” includes reference to curative, palliative and prophylactic treatment.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

### Polypeptides, Polynucleotides, and Vectors

In one aspect, the present disclosure provides an isolated polypeptide which comprises an N-terminal portion of Apolipoprotein B100 (ApoB100), or a functional derivative thereof. In some embodiments, the isolated polypeptide which comprises an N-terminal portion of Apolipoprotein B100 (ApoB100), or a functional derivative thereof, does not comprise any ApoB100 sequences beyond amino acid residue 816 of ApoB100 (see, e.g., SEQ ID NO: 1). In some embodiments, the ApoB100 has the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with SEQ ID NO: 1.

In some embodiments, the isolated polypeptide does not comprise amino acid residue 816 of ApoB100 (SEQ ID NO: 1). In some embodiments, the isolated polypeptide comprises amino acid residues 363-544 of ApoB100 (SEQ ID NO: 1), or a derivative thereof. In some embodiments, the isolated polypeptide comprises amino acid residues 1-544 of ApoB100, or a derivative thereof. In some embodiments, the isolated polypeptide comprises amino acid residues 544-684 of ApoB100, or a derivative thereof. In some embodiments, the isolated polypeptide comprises amino acid residues 363-684 of ApoB100, or a derivative thereof. In some embodiments, the isolated polypeptide comprises amino acid residues 1-684 of ApoB100, or a derivative thereof.

In some embodiments, the isolated polypeptide comprising the N-terminal portion of Apolipoprotein B100 (ApoB100), or the functional derivative thereof, is capable of inhibiting scavenger receptor-B1 (SR-B1). In some embodiments, the isolated polypeptide is capable of inhibiting activin receptor-like kinase 1 (ALK1). In some embodiments, the isolated polypeptide is capable of inhibiting scavenger receptor-B1 (SR-B1) and/or activin receptor-like kinase 1 (ALK1).

In some embodiments, scavenger receptor-B1 (SR-B1) may be inhibited by about 50% or more. The scavenger receptor-B1 (SR-B1) may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. Scavenger receptor-B1 (SR-B1) may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, activin receptor-like kinase 1 (ALK1) may be inhibited by about 50% or more. Activin receptor-like kinase 1 (ALK1) may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. Activin receptor-like kinase 1 (ALK1) may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, scavenger receptor-B1 (SR-B1) and/or activin receptor-like kinase 1 (ALK1) may, together or separately, be inhibited by about 50% or more. Scavenger receptor-B1 (SR-B1) and/or activin receptor-like kinase 1 (ALK1) may, together or separately, be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. Scavenger receptor-B1 (SR-B1) and/or activin receptor-like kinase 1 (ALK1) may, together or separately, be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the isolated polypeptide comprising the N-terminal portion of Apolipoprotein B100 (ApoB100), or the functional derivative thereof, comprises amino acid residues 363-544 of ApoB100, or a derivative thereof. In some embodiments, the isolated polypeptide comprising the N-terminal portion of Apolipoprotein B100 (ApoB100), or the functional derivative thereof, consists of amino acid residues 363-544 of ApoB100, or a derivative thereof.

In some embodiments, the isolated polypeptide comprising the N-terminal portion of Apolipoprotein B100 (ApoB100), or the functional derivative thereof, comprises amino acid residues 363-544 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 1. In some embodiments, the nucleotide sequence that encodes the N-terminal portion of Apolipoprotein B100 (ApoB100), or the functional derivative thereof, comprising amino acid residues 363-544 of the amino acid sequence of SEQ ID NO: 1, comprises the nucleotide sequence that encodes amino acid residues 363-544 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 363-544 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 363-544 of the amino acid sequence of SEQ ID NO: 1.

In some embodiments, the isolated polypeptide comprising the N-terminal portion of Apolipoprotein B100 (ApoB100), or the functional derivative thereof, comprises an amino acid sequence that has at least 80% sequence identity to amino acid residues 363-544 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 363-544 of the amino acid sequence of SEQ ID NO: 1, or a derivative thereof. In some embodiments, the isolated polypeptide comprising the N-terminal portion of Apolipoprotein B100 (ApoB100), or the functional derivative thereof, consists of amino acid residues 363-544 of the amino acid sequence of SEQ ID NO: 1, or a derivative thereof.

In some embodiments, the isolated polypeptide comprising the N-terminal portion of Apolipoprotein B100 (ApoB100), or the functional derivative thereof, comprises the amino acid sequence of SEQ ID NO: 5, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 5. In some embodiments, the nucleotide sequence that encodes the isolated polypeptide comprising the N-terminal portion of Apolipoprotein B100 (ApoB100), or the functional derivative thereof, comprises the nucleotide sequence that encodes the amino acid sequence of SEQ ID NO: 5, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 5. In some embodiments, the isolated polypeptide comprising the N-terminal portion of Apolipoprotein B100 (ApoB100), or the functional derivative thereof, comprises the amino acid sequence of SEQ ID NO: 5.

In some embodiments, the isolated polypeptide comprising the N-terminal portion of Apolipoprotein B100 (ApoB100), or the functional derivative thereof, comprises an amino acid sequence that has at least 80% sequence identity to the amino acid sequence of SEQ ID NO: 5. In some embodiments, the isolated polypeptide comprising the N-terminal portion of Apolipoprotein B100 (ApoB100), or the functional derivative thereof, comprises the amino acid sequence of SEQ ID NO: 5, or a derivative thereof. In some embodiments, the isolated polypeptide comprising the N-terminal portion of Apolipoprotein B100 (ApoB100), or the functional derivative thereof, consists of the amino acid sequence of SEQ ID NO: 5, or a derivative thereof.

In some embodiments, the isolated polypeptide comprising the N-terminal portion of Apolipoprotein B100 (ApoB100), or the functional derivative thereof, comprises amino acid residues 1-544 of ApoB100, or a derivative thereof. In some embodiments, the isolated polypeptide comprising the N-terminal portion of Apolipoprotein B100 (ApoB100), or the functional derivative thereof, consists of amino acid residues 1-544 of ApoB100, or a derivative thereof.

In some embodiments, the isolated polypeptide comprising the N-terminal portion of Apolipoprotein B100 (ApoB100), or the functional derivative thereof, comprises amino acid residues 1-544 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 1-544 of the amino acid sequence of SEQ ID NO: 1. In some embodiments, the nucleotide sequence that encodes the isolated polypeptide comprising the N-terminal portion of Apolipoprotein B100 (ApoB100), or the functional derivative thereof, comprising amino acid residues 1-544 of the amino acid sequence of SEQ ID NO: 1, comprises the nucleotide sequence that encodes amino acid residues 1-544 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 1-544 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 1-544 of the amino acid sequence of SEQ ID NO: 1.

In some embodiments, the isolated polypeptide comprising the N-terminal portion of Apolipoprotein B100 (ApoB100), or the functional derivative thereof, comprises an amino acid sequence that has at least 80% sequence identity to amino acid residues 1-544 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 1-544 of the amino acid sequence of SEQ ID NO: 1, or a derivative thereof. In some embodiments, the isolated polypeptide comprising the N-terminal portion of Apolipoprotein B100 (ApoB100), or the functional derivative thereof, consists of amino acid residues 1-544 of the amino acid sequence of SEQ ID NO: 1, or a derivative thereof.

In some embodiments, the isolated polypeptide comprising the N-terminal portion of Apolipoprotein B100 (ApoB100), or the functional derivative thereof, comprises the amino acid sequence of SEQ ID NO: 4, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 4. In some embodiments, the nucleotide sequence that encodes the N-terminal portion of Apolipoprotein B100 (ApoB100), or the functional derivative thereof, comprises the nucleotide sequence that encodes the amino acid sequence of SEQ ID NO: 4, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 4. In some embodiments, the isolated polypeptide comprising the N-terminal portion of Apolipoprotein B100 (ApoB100), or the functional derivative thereof, comprises the amino acid sequence of SEQ ID NO: 4.

In some embodiments, the isolated polypeptide comprising the N-terminal portion of Apolipoprotein B100 (ApoB100), or the functional derivative thereof, comprises an amino acid sequence that has at least 80% sequence identity to the amino acid sequence of SEQ ID NO: 4. In some embodiments, the isolated polypeptide comprising the N-terminal portion of Apolipoprotein B100 (ApoB100), or the functional derivative thereof, comprises the amino acid sequence of SEQ ID NO: 4, or a derivative thereof. In some embodiments, the isolated polypeptide comprising the N-terminal portion of Apolipoprotein B100 (ApoB100), or the functional derivative thereof, consists of the amino acid sequence of SEQ ID NO: 4, or a derivative thereof.

In some embodiments, the isolated polypeptide comprising the N-terminal portion of Apolipoprotein B100 (ApoB100), or the functional derivative thereof, comprises amino acid residues 544-684 of ApoB100, or a derivative thereof. In some embodiments, the isolated polypeptide comprising the N-terminal portion of Apolipoprotein B100 (ApoB100), or the functional derivative thereof, consists of amino acid residues 544-684 of ApoB100, or a derivative thereof.

In some embodiments, the isolated polypeptide comprising the N-terminal portion of Apolipoprotein B100 (ApoB100), or the functional derivative thereof, comprises amino acid residues 544-684 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 544-684 of the amino acid sequence of SEQ ID NO: 1. In some embodiments, the nucleotide sequence that encodes the isolated polypeptide comprising the N-terminal portion of Apolipoprotein B100 (ApoB100), or the functional derivative thereof, comprising amino acid residues 544-684 of the amino acid sequence of SEQ ID NO: 1, comprises the nucleotide sequence that encodes amino acid residues 544-684 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 544-684 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 544-684 of the amino acid sequence of SEQ ID NO: 1.

In some embodiments, the isolated polypeptide comprising the N-terminal portion of Apolipoprotein B100 (ApoB100), or the functional derivative thereof, comprises an amino acid sequence that has at least 80% sequence identity to amino acid residues 544-684 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 544-684 of the amino acid sequence of SEQ ID NO: 1, or a derivative thereof. In some embodiments, the isolated polypeptide comprising the N-terminal portion of Apolipoprotein B100 (ApoB100), or the functional derivative thereof, consists of amino acid residues 544-684 of the amino acid sequence of SEQ ID NO: 1, or a derivative thereof.

In some embodiments, the isolated polypeptide comprising the N-terminal portion of Apolipoprotein B100 (ApoB100), or the functional derivative thereof, comprises the amino acid sequence of SEQ ID NO: 7, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 7. In some embodiments, the nucleotide sequence that encodes the N-terminal portion of Apolipoprotein B100 (ApoB100), or the functional derivative thereof, comprises the nucleotide sequence that encodes the amino acid sequence of SEQ ID NO: 7, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 7. In some embodiments, the isolated polypeptide comprising the N-terminal portion of Apolipoprotein B100 (ApoB100), or the functional derivative thereof, comprises the amino acid sequence of SEQ ID NO: 7.

In some embodiments, the isolated polypeptide comprising the N-terminal portion of Apolipoprotein B100 (ApoB100), or the functional derivative thereof, comprises an amino acid sequence that has at least 80% sequence identity to the amino acid sequence of SEQ ID NO: 7. In some embodiments, the isolated polypeptide comprising the N-terminal portion of Apolipoprotein B100 (ApoB100), or the functional derivative thereof, comprises the amino acid sequence of SEQ ID NO: 7, or a derivative thereof. In some embodiments, the isolated polypeptide comprising the N-terminal portion of Apolipoprotein B100 (ApoB100), or the functional derivative thereof, consists of the amino acid sequence of SEQ ID NO: 7, or a derivative thereof.

In some embodiments, the isolated polypeptide comprising the N-terminal portion of Apolipoprotein B100 (ApoB100), or the functional derivative thereof, comprises amino acid residues 363-684 of ApoB100, or a derivative thereof. In some embodiments, the isolated polypeptide comprising the N-terminal portion of Apolipoprotein B100 (ApoB100), or the functional derivative thereof, consists of amino acid residues 363-684 of ApoB100, or a derivative thereof.

In some embodiments, the isolated polypeptide comprising the N-terminal portion of Apolipoprotein B100 (ApoB100), or the functional derivative thereof, comprises amino acid residues 363-684 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 363-684 of the amino acid sequence of SEQ ID NO: 1. In some embodiments, the nucleotide sequence that encodes the isolated polypeptide comprising the N-terminal portion of Apolipoprotein B100 (ApoB100), or the functional derivative thereof, comprising amino acid residues 363-684 of the amino acid sequence of SEQ ID NO: 1, comprises the nucleotide sequence that encodes amino acid residues 363-684 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 363-684 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 363-684 of the amino acid sequence of SEQ ID NO: 1.

In some embodiments, the isolated polypeptide comprising the N-terminal portion of Apolipoprotein B100 (ApoB100), or the functional derivative thereof, comprises an amino acid sequence that has at least 80% sequence identity to amino acid residues 363-684 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 363-684 of the amino acid sequence of SEQ ID NO: 1, or a derivative thereof. In some embodiments, the isolated polypeptide comprising the N-terminal portion of Apolipoprotein B100 (ApoB100), or the functional derivative thereof, consists of amino acid residues 363-684 of the amino acid sequence of SEQ ID NO: 1, or a derivative thereof.

In some embodiments, the isolated polypeptide comprising the N-terminal portion of Apolipoprotein B100 (ApoB100), or the functional derivative thereof, comprises the amino acid sequence of SEQ ID NO: 6, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 6. In some embodiments, the nucleotide sequence that encodes the N-terminal portion of Apolipoprotein B100 (ApoB100), or the functional derivative thereof, comprises the nucleotide sequence that encodes the amino acid sequence of SEQ ID NO: 6, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 6. In some embodiments, the isolated polypeptide comprising the N-terminal portion of Apolipoprotein B100 (ApoB100), or the functional derivative thereof, comprises the amino acid sequence of SEQ ID NO: 6.

In some embodiments, the isolated polypeptide comprising the N-terminal portion of Apolipoprotein B100 (ApoB100), or the functional derivative thereof, comprises an amino acid sequence that has at least 80% sequence identity to the amino acid sequence of SEQ ID NO: 6. In some embodiments, the isolated polypeptide comprising the N-terminal portion of Apolipoprotein B100 (ApoB100), or the functional derivative thereof, comprises the amino acid sequence of SEQ ID NO: 6, or a derivative thereof. In some embodiments, the isolated polypeptide comprising the N-terminal portion of Apolipoprotein B100 (ApoB100), or the functional derivative thereof, consists of the amino acid sequence of SEQ ID NO: 6, or a derivative thereof.

In some embodiments, the isolated polypeptide comprising the N-terminal portion of Apolipoprotein B100 (ApoB100), or the functional derivative thereof, comprises amino acid residues 1-684 of ApoB100, or a derivative thereof. In some embodiments, the isolated polypeptide comprising the N-terminal portion of Apolipoprotein B100 (ApoB100), or the functional derivative thereof, consists of amino acid residues 1-684 of ApoB100, or a derivative thereof.

In some embodiments, the isolated polypeptide comprising the N-terminal portion of Apolipoprotein B100 (ApoB100), or the functional derivative thereof, comprises amino acid residues 1-684 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 1-684 of the amino acid sequence of SEQ ID NO: 1. In some embodiments, the nucleotide sequence that encodes the isolated polypeptide comprising the N-terminal portion of Apolipoprotein B100 (ApoB100), or the functional derivative thereof, comprising amino acid residues 1-684 of the amino acid sequence of SEQ ID NO: 1, comprises the nucleotide sequence that encodes amino acid residues 1-684 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 1-684 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 1-684 of the amino acid sequence of SEQ ID NO: 1.

In some embodiments, the isolated polypeptide comprising the N-terminal portion of Apolipoprotein B100 (ApoB100), or the functional derivative thereof, comprises an amino acid sequence that has at least 80% sequence identity to amino acid residues 1-684 of the amino acid sequence of SEQ ID NO: 1. In some embodiments, the isolated polypeptide comprising the N-terminal portion of Apolipoprotein B100 (ApoB100), or the functional derivative thereof, comprises amino acid residues 1-684 of the amino acid sequence of SEQ ID NO: 1, or a derivative thereof. In some embodiments, the isolated polypeptide comprising the N-terminal portion of Apolipoprotein B100 (ApoB100), or the functional derivative thereof, consists of amino acid residues 1-684 of the amino acid sequence of SEQ ID NO: 1, or a derivative thereof.

In some embodiments, the isolated polypeptide comprising the N-terminal portion of Apolipoprotein B100 (ApoB100), or the functional derivative thereof, comprises the amino acid sequence of SEQ ID NO: 3, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 3. In some embodiments, the nucleotide sequence that encodes the N-terminal portion of Apolipoprotein B100 (ApoB100), or the functional derivative thereof, comprises the nucleotide sequence that encodes the amino acid sequence of SEQ ID NO: 3, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 3. In some embodiments, the isolated polypeptide comprising the N-terminal portion of Apolipoprotein B100 (ApoB100), or the functional derivative thereof, comprises the amino acid sequence of SEQ ID NO: 3.

In some embodiments, the isolated polypeptide comprising the N-terminal portion of Apolipoprotein B100 (ApoB100), or the functional derivative thereof, comprises an amino acid sequence that has at least 80% sequence identity to the amino acid sequence of SEQ ID NO: 3. In some embodiments, the isolated polypeptide comprising the N-terminal portion of Apolipoprotein B100 (ApoB100), or the functional derivative thereof, comprises the amino acid sequence of SEQ ID NO: 3, or a derivative thereof. In some embodiments, the isolated polypeptide comprising the N-terminal portion of Apolipoprotein B100 (ApoB100), or the functional derivative thereof, consists of the amino acid sequence of SEQ ID NO: 3, or a derivative thereof.

A polynucleotide comprising a nucleotide sequence encoding any of the polypeptides disclosed herein is also within the scope of the present disclosure. In some embodiments, the polynucleotides may comprise any of various nucleotides described herein. In various embodiments, the polynucleotide is RNA. In some embodiments, the RNA is mRNA. In various embodiments, the polynucleotide is DNA.

In various embodiments, polynucleotide compositions described herein may be incorporated, e.g., into a lipid nanoparticle(s), liposome(s), or non-lipid nanoparticle(s). In some embodiments, a polynucleotide described herein is incorporated into liposomes. In some embodiments, polynucleotide compositions of the present disclosure may comprise (e.g., may be complexed to), e.g., liposomes, micelles, nanodisperse albumin and its modifications, polymer nanoparticles, dendrimers, and/or inorganic nanoparticles of different compositions.

In one aspect, the present disclosure provides a recombinant vector comprising a polynucleotide described herein. In some embodiments, the vector is a viral vector. Suitable viral vectors that can be used in the present disclosure include, but are not limited to, an adenoviral vector, a baculoviral vector, an adeno-associated viral (AAV) vector, a herpes viral vector, a retroviral vector, and/or an adeno-associated viral (AAV) vector. In some embodiments, the viral vector is an adenovirus. In some embodiments, the viral vector is a lentiviral vector. In some embodiments, the viral vector is an adeno-associated viral (AAV) vector. In some embodiments the AAV vector is selected from, without limitation, AAV1, AAV2,AAV3, AAV3B, AAV4, AAV5,AAV6, AAV6.2, AAV7, AAV8, AAV9, AAV10, AAV11,AAV type hull (AAV hull), AAV12, AAV13, AAVDJ, AAV retro (AAV retro), Anc80L65, AAVLK03, AAV type rh32.33 (AAVrh.32.33), AAV PUPS, AAV PP.B, AAV PHP.eB, AAVrh.64R1, AAVhu.37, AAVrh.8, and AAV2/8,AAV2G9. In some embodiments, the AAV vector is AAV8 vector. In some embodiments, the AAV vector is AAV2 vector.

In some embodiments, the viral vector has tropism for the liver.

In some embodiments, the vector is a non-viral vector. In some embodiments, the non-viral vector is a transposon such as, but are not limited to, a PiggyBac transposon and a sleeping beauty transposon.

In some embodiments, the vector is a plasmid.

In various embodiments, a recombinant vector comprises a polynucleotide described herein, and the polynucleotide may be operably linked to one or more regulatory sequences for expression of the polypeptide. Examples of regulatory sequences include transcription initiation, promoter, terminator, and enhancer sequences. In some embodiments, the polynucleotide is operably linked to a promoter. The promoter may be a constitutive promoter or inducible promoter.

### Pharmaceutical Compositions

Any of isolated polypeptides and/or related polynucleotides or recombinant vectors described herein can be present in a pharmaceutical composition (such as a formulation) that can includes other agents, excipients, or stabilizers. In various embodiments, a pharmaceutical composition described herein may comprise (i) a polypeptide described herein, (ii) a polynucleotide described herein, and/or (iii) a recombinant vector described herein, and a pharmaceutically acceptable carrier or adjuvant.

It is understood that the compounds of the present disclosure can be present in one or more stereoisomers (e.g., diastereomers). The disclosure includes, within its scope, all of these stereoisomers, either isolated (e.g., in enantiomeric isolation) or in combination (including racemic and diastereomeric mixtures). The present disclosure uses amino acids independently selected from L and D forms (e.g., the peptide may contain two serine residues, each serine residue having the same or opposite absolute stereochemistry), etc., are intended for the use of both L- and D-form amino acids.

Accordingly, the compounds of the present disclosure also include substantially pure stereoisomeric form of the specific compound with respect to the asymmetric center of the amino acid residue, for example about 90% de, such as greater than about 95% to 97% de, or 99% de. For larger compounds, as well as mixtures thereof (such as racemic mixtures). Such diastereomers may be prepared, for example, by asymmetric synthesis using chiral intermediates, or the mixture may be divided by conventional methods, such as chromatography or the use of dividing agents.

If the compounds of the disclosure require purification, chromatographic techniques such as high-performance liquid chromatography (HPLC) and reverse phase HPLC can be used. Peptides may be characterized by mass spectrometry and/or other suitable methods.

If the compound contains one or more functional groups that can be protonated or deprotonated (e.g., at physiological pH), the compound can be prepared and/or isolated as a pharmaceutically acceptable salt. It will be appreciated that the compound can be zwitterion at a given pH. As used herein, the expression “pharmaceutically acceptable salt” refers to a salt of a given compound, which salt is suitable for pharmaceutical administration. Such salts can be formed, for example, by reacting an acid or base with an amine or carboxylic acid group, respectively.

Pharmaceutically acceptable acid addition salts can be prepared from inorganic and organic acids. Examples of inorganic acids include hydrochloric acid, hydrobromic acid, sulfuric acid, nitric acid, phosphoric acid and the like. Examples of organic acids include acetic acid, propionic acid, glycolic acid, pyruvate, oxalic acid, malic acid, malonic acid, succinic acid, maleic acid, fumaric acid, tartrate acid, citrate, benzoic acid, cinnamic acid, mandelic acid, Examples thereof include methanesulfonic acid, ethanesulfonic acid, p-toluenesulfonic acid and salicylic acid.

Pharmaceutically acceptable base addition salts can be prepared from inorganic and organic bases. Corresponding counterions derived from inorganic bases include salts of sodium, potassium, lithium, ammonium, calcium and magnesium. Organic bases include isopropylamine, trimethylamine, diethylamine, triethylamine, tripropylamine, ethanolamine, 2-dimethylaminoethanol, tromethamine, lysine, arginine, histidine, caffeine, prokine, hydrabamine, choline, betaine, ethylenediamine, glucosamine, Substituted amines such as primary, secondary and tertiary amines such as N-alkylglucamine, theobromine, purines, piperazine, piperazine and N-ethylpiperidine, substituted amines such as natural substituted amines and cyclic amines can be mentioned.

Acid/base addition salts tend to be more soluble in aqueous solvents than the corresponding free acid/base forms.

In some embodiments, it is envisioned that two or more combinations of the compounds of the disclosure will be administered to the subject. It is believed that the compound (s) may also be administered in combination with one or more additional therapeutic agents. This combination can allow separate, continuous or simultaneous administration with the other active ingredients of the above compounds. This combination may be provided in the form of a pharmaceutical composition.

As used herein, the term “combination” is used by the combination agents as defined above dependently or independently, or by the use of different fixed combinations with different amounts of combination agents, i.e. simultaneously or at different times. Refers to a kit of compositions or parts that can be administered. The combination agents can then be administered, for example, simultaneously or staggered in time (i.e., at different times and at equal or different time intervals for any part of the kit). The ratio of the total amount of combination agents administered in a combination can vary, e.g., to address the needs of a subpopulation of patients to be treated or the needs of a single patient, and different needs are the age of the patient, it can be due to gender, weight, etc.

The route of administration and the type of pharmaceutically acceptable carrier will depend on the condition being treated and the type of mammal. Formulations containing the active compound may be prepared such that the activity of the compound is not disrupted during the process and the compound can reach its site of action without disruption. In some cases, it may be necessary to protect the compound by means known in the art, such as microencapsulation. Similarly, the route of dosing selected should be such that the compound reaches its site of action.

In some embodiments, the composition further comprises a targeting agent or a carrier that promotes the delivery of the inhibitors of endocytosis to an area affected by the chronic pain. Exemplary carriers include liposomes, micelles, nanodisperse albumin and its modifications, polymer nanoparticles, dendrimers, inorganic nanoparticles of different compositions.

The appropriate formulation for the compound of the disclosure can be adjusted for pH. Buffer systems are routinely used to provide pH values in the desired range and include carboxylic acid buffers such as acetates, citrates, lactates and succinates. In some embodiments, the composition is formulated to have a pH range of about 4.5 to about 9.0, including for example pH ranges of about any of 5.0 to about 8.0, about 6.5 to about 7.5, and about 6.5 to about 7.0. In some embodiments, the pH of the composition is formulated to no less than about 6, including for example no less than about any of 6.5, 7, or 8 (such as about 8). The composition can also be made to be isotonic with blood by the addition of a suitable tonicity modifier, such as glycerol.

The formulation may also include suitable excipients, such as antioxidants. Examples of antioxidants include phenolic compounds such as BHT or Vitamin E, reducing agents such as methionine or sulfites, and metal chelating agents such as EDTA.

The compounds or pharmaceutically acceptable salts thereof described herein can be prepared in parenteral dosage forms such as those suitable for, e.g., intravascular (intravenous or intraarterial), intraperitoneal, intratumoral, intraventricular, intrapleural or intramuscular administration delivery. Suitable pharmaceutical forms for injectable use include sterile injectable or dispersions and sterile powders for the immediate preparation of sterile injectable solutions. They must be stable under manufacturing and storage conditions and protected from reduction or oxidation and the contaminating effects of microorganisms such as bacteria or fungi.

The solvent or dispersion medium for the injectable solution or dispersion may include either conventional solvents or carrier systems for the active compound, e.g., water, ethanol, polyols (e.g., glycerol, propylene glycol and). Liquid polyethylene glycol, etc., suitable mixtures thereof, and vegetable oils may be included. Proper fluidity can be maintained, for example, by the use of a coating such as lecithin, the maintenance of the required particle size in the case of dispersions, and the use of surfactants. Prevention of the action of microorganisms can be performed as needed by incorporating various antibacterial and antifungal agents such as parabens, chlorobutanol, phenol, sorbic acid, thimerosal and the like. In many cases, it may be preferable to include agents that regulate osmotic pressure, such as sugar or sodium chloride. Preferably, the injectable formulation is isotonic with blood. Sustained absorption of the injectable composition can be brought about by the use of agents that delay absorption (e.g., aluminum monostearate and gelatin) in the composition. Suitable pharmaceutical forms for injection can be delivered by any suitable route, including intravenous, intramuscular, intracerebral, intrathecal, epidural injection or infusion.

Sterilized injectable solutions are prepared by adding the required amount of the compounds of the disclosure to a suitable solvent containing various other components, such as those listed above, as needed, followed by filtration sterilization. Generally, dispersions are prepared by incorporating various sterile active ingredients into a sterile vehicle containing a basic dispersion medium and other required ingredients from those described above. For sterile powders for the preparation of sterile injectable solutions, the preferred method of preparation is vacuum drying or lyophilization of the pre-sterile filtered solution of the active ingredient plus any additional desired ingredients.

Other pharmaceutical forms include the oral and enteral formulations, where the active compound can be formulated with an inert diluent or an assimilated edible carrier, or encapsulated in hard or softshell gelatin capsules. The formulations can also be tableted, or it can be incorporated directly into diet foods. For oral therapeutic administration, the active compound is taken up with excipients and used in the form of ingestible tablets, buccal or sublingual tablets, troches, capsules, elixirs, suspensions, syrups, wafers, etc. The amount of active compound in such a therapeutically useful composition is such that an appropriate dose can be obtained.

Tablets, lozenges, pills, capsules, etc. may also contain the ingredients listed below: binders such as gum, acacia, corn starch or gelatin; excipients such as dicalcium phosphate; corn starch, Disintegrants such as potato starch, arginic acid; lubricants such as magnesium stearate; sweeteners such as sucrose, lactose or saccharin, or flavors such as peppermint, winter green oil, or cherry flavor may be added. If the dosage unit form is a capsule, it may contain a liquid carrier in addition to the above types of materials. Various other materials may be present as a coating or in other ways to alter the physical form of the dosage unit. For example, tablets, pills, or capsules can be coated with shellac, sugar, or both. The syrup or elixir may contain active compounds, sucrose as a sweetener, methyl and propylparabens as preservatives, pigments and flavors such as cherry or orange flavors. Of course, any substance used to prepare the dosage unit form must be pharmaceutically pure and substantially non-toxic in the amount used. In addition, the compounds of the disclosure may be incorporated into sustained release formulations and formulations comprising those that specifically deliver the active peptide to a particular region of the intestine.

Liquid formulations can also be administered enterally via the stomach or esophageal canal. The enteral preparation can be prepared in the form of a suppository by mixing with a suitable base such as an emulsifying base or a water-soluble base. It is possible, but not necessary, to administer the compound of the present disclosure topically, intranasally, intravaginally, intraocularly or the like.

Pharmaceutically acceptable vehicles and/or diluents include any and all solvents, dispersion media, coatings, antibacterial and antifungal agents, isotonic and absorption retarders, and the like. The use of such vehicles and agents for pharmaceutically active substances is well known in the art. Its use in therapeutic compositions is intended unless any conventional vehicle or agent is incompatible with the active ingredient. Auxiliary active ingredients can also be incorporated into the composition.

It is particularly advantageous to formulate the composition in unit dosage form for ease of administration and uniformity of dosage. As used herein, a dosage unit form means a physically distinct unit suitable as a unit dosage for a mammalian subject to be treated; each unit is a required pharmaceutically acceptable vehicle. A dosage unit form may contain a predetermined amount of active substance calculated to produce a desired therapeutic effect described herein. Details of the novel dosage unit forms of the disclosure include (a) the unique properties of the active substance and the particular therapeutic effect to be achieved, and (b) physical health as disclosed in detail herein. It is determined by and directly dependent on the technology-specific limitations of the active substances formulated for the treatment of the disease in living subjects with impaired disease states.

As mentioned above, the main active ingredient may be formulated for convenient and effective administration in therapeutically effective amounts using a suitable pharmaceutically acceptable vehicle in the form of a dosage unit. The unit dosage form can contain, for example, the major active compound in an amount ranging from 0.25 μg to about 2000 mg. Expressed in proportion, the active compound may be present in a carrier of about 0.25 μg to about 2000 mg/mL. In the case of a composition containing an auxiliary active ingredient, the dose is determined with reference to the usual dosage and mode of administration of the ingredient.

In some embodiments, the composition is suitable for administration to a human. In some embodiments, the composition is suitable for administration to a mammal such as, in the veterinary context, domestic pets and agricultural animals. There are a wide variety of suitable formulations of the composition comprising the inhibitor of endocytosis. The following formulations and methods are merely exemplary and are in no way limiting. Formulations suitable for oral administration can consist of (a) liquid solutions, such as an effective amount of the compound dissolved in diluents, such as water, saline, or orange juice, (b) capsules, sachets or tablets, each containing a predetermined amount of the active ingredient, as solids or granules, (c) suspensions in an appropriate liquid, and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such excipients as are known in the art.

Examples of suitable carriers, excipients, and diluents include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline solution, syrup, methylcellulose, methyl and propylhydroxybenzoates, talc, magnesium stearate, and mineral oil. In some embodiments, the composition comprising the inhibitor of endocytosis with a carrier as discussed herein is present in a dry formulation (such as lyophilized composition). The formulations can additionally include lubricating agents, wetting agents, emulsifying and suspending agents, preserving agents, sweetening agents or flavoring agents.

Formulations suitable for parenteral administration include aqueous and non-aqueous, isotonic sterile injection solutions, which can contain anti-oxidants, buffers, bacteriostats, and solutes that render the formulation compatible with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

### Methods of Use

The pharmaceutical compositions comprising, e.g., polypeptide(s), polynucleotide(s), and/or vector(s), described herein and, e.g., a carrier and/or excipient disclosed herein, may be used for various therapeutic applications (in vivo and ex vivo) and as research tools.

In one aspect, the present disclosure provides a method of treating or preventing atherosclerosis and/or an associated disease, disorder, or condition thereof in a subject in need thereof, comprising administering to the subject a therapeutically effective amount of an inhibitor of scavenger receptor-B1 (SR-B1) and/or activin receptor-like kinase 1 (ALK1). In some embodiments, the disease, disorder, or condition associated with atherosclerosis is an inflammatory disease, obesity, metabolic disease, or eruptive xanthomas. In some embodiments, the subject is human.

In some embodiments, the inhibitor of scavenger receptor-B1 (SR-B1) may inhibit scavenger receptor-B1 (SR-B1) by about 50% or more. In some embodiments, the inhibitor of scavenger receptor-B1 (SR-B1) may inhibit scavenger receptor-B1 (SR-B1) by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The inhibitor of scavenger receptor-B1 (SR-B1) may inhibit scavenger receptor-B1 (SR-B1) by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the inhibitor of activin receptor-like kinase 1 (ALK1) may inhibit activin receptor-like kinase 1 (ALK1) by about 50% or more. In some embodiments, the inhibitor of activin receptor-like kinase 1 (ALK1) may inhibit activin receptor-like kinase 1 (ALK1) by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The inhibitor of activin receptor-like kinase 1 (ALK1) may inhibit activin receptor-like kinase 1 (ALK1) by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the inhibitor(s) can inhibit the interaction between ApoB100 with SR-B1 and/or ALK1. In some embodiments, the inhibitor(s) can inhibit the interaction between ApoB100 with SR-B1 and/or ALK1 by about 50% or more. In some embodiments, the inhibitor(s) can inhibit the interaction between ApoB100 with SR-B1 and/or ALK1 by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The inhibitor(s) can inhibit the interaction between ApoB100 with SR-B1 and/or ALK1 by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments of any of the methods of treating or preventing atherosclerosis and/or an associated disease, disorder, or condition thereof in a subject in need thereof described herein, the inhibitor of SR-B1 and/or ALK1 may comprise (i) a polypeptide described herein; (ii) a polynucleotide described herein; (iii) a recombinant vector described herein; and/or (iv) a pharmaceutical composition described herein.

In some embodiments, the inhibitor of SR-B1 and/or ALK1 comprises a polypeptide consisting of amino acid residues 1-816 of ApoB100, or a derivative thereof; a polynucleotide encoding the polypeptide or derivative thereof; a recombinant vector comprising the polynucleotide; or a pharmaceutical composition comprising the polypeptide or derivative thereof, the polynucleotide, or the recombinant vector.

In some embodiments, the inhibitor of SR-1 and/or ALK1 comprises a polypeptide comprising amino acid residues 1-816 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 1-816 of the amino acid sequence of SEQ ID NO: 1. In some embodiments, the nucleotide sequence that encodes the inhibitor of SR-1 and/or ALK1 comprising a polypeptide that comprises amino acid residues 1-816 of the amino acid sequence of SEQ ID NO: 1, comprises the nucleotide sequence that encodes amino acid residues 1-816 of the amino acid sequence of SEQ ID NO: 1, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with amino acid residues 1-816 of the amino acid sequence of SEQ ID NO: 1. In some embodiments, the inhibitor of SR-B1 and/or ALK1 comprises a polypeptide comprising amino acid residues 1-816 of the amino acid sequence of SEQ ID NO: 1, or a derivative thereof. In some embodiments, the inhibitor of SR-B1 and/or ALK1 comprises a polypeptide consisting of amino acid residues 1-816 of the amino acid sequence of SEQ ID NO: 1, or a derivative thereof.

In some embodiments, the inhibitor of SR-B1 and/or ALK1 comprises a polypeptide comprising the amino acid sequence of SEQ ID NO: 2, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 2. In some embodiments, the nucleotide sequence that encodes the inhibitor of SR-B1 and/or ALK1 comprising a polypeptide that comprises the amino acid sequence of SEQ ID NO: 2, comprises the nucleotide sequence that encodes the amino acid sequence of SEQ ID NO: 2, or a variant thereof having at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99%, sequence identity with the amino acid sequence of SEQ ID NO: 2. In some embodiments, the inhibitor of SR-B1 and/or ALK1 comprises a polypeptide comprising the amino acid sequence of SEQ ID NO: 2, or a derivative thereof. In some embodiments, the inhibitor of SR-B1 and/or ALK1 comprises a polypeptide consists of the amino acid sequence of SEQ ID NO: 2, or a derivative thereof.

In some embodiments, the inhibitor of SR-B1 and/or ALK1 is a small molecule, an antibody, an siRNA, an shRNA, or an antisense oligonucleotide.

In some embodiments, the small molecule is a synthetic compound or a plant derived compound.

In some embodiments, the inhibitor is administered intravenously, subcutaneously, orally, or intranasally.

In some embodiments, the inhibitor is administered with an additional agent. A non-limiting example of an additional agent is semaglutide.

The present disclosure further encompasses a method of blocking uptake and/or transcytosis of low-density lipoprotein (LDL), very-low-density lipoprotein (VLDL), chylomicrons, and/or lipoprotein (a) (Lp(a)) by an endothelial cell. The method comprises contacting the endothelial cell with an effective amount of an inhibitor of scavenger receptor-B1 (SR-B1) and/or activin receptor-like kinase 1 (ALK1). In some embodiments, the inhibitor of scavenger receptor-B1 (SR-B1) and/or activin receptor-like kinase 1 (ALK1) may comprise any of various polypeptides and/or related polynucleotides and/or vectors, or pharmaceutical compositions thereof, of the present disclosure. In some embodiments, the endothelial cell is an aortic endothelial cell. In some embodiments, the cell is in a subject and the inhibitor of SR-B1 and/or ALK1 is administered to the subject. In some embodiments, the subject has atherosclerosis and/or an associated disease, disorder, or condition thereof. In some embodiments, the subject is at high risk of developing atherosclerosis and/or an associated disease, disorder, or condition thereof. In some embodiments, the inhibitor(s) can inhibit the interaction between ApoB100 with SR-B1 and/or ALK1. In some embodiments, the inhibitor(s) can inhibit the interaction between ApoB100 with SR-1 and/or ALK1. In some embodiments, the inhibitor(s) can inhibit the interaction between ApoB100 with SR-1 and/or ALK1 by about 50% or more. In some embodiments, the inhibitor(s) can inhibit the interaction between ApoB100 with SR-n1 and/or ALK1 by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The inhibitor(s) can inhibit the interaction between ApoB100 with SR-B1 and/or ALK1 by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments of any of the above-described methods, the uptake and/or transcytosis of low-density lipoprotein (LDL), very-low-density lipoprotein (VLDL), chylomicrons, and/or lipoprotein (a) (Lp(a)) by an endothelial cell is blocked by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The uptake and/or transcytosis of low-density lipoprotein (LDL), very-low-density lipoprotein (VLDL), chylomicrons, and/or lipoprotein (a) (Lp(a)) by an endothelial cell may be blocked by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

### EXAMPLES

The following examples are provided to further describe some of the embodiments disclosed herein. The examples are intended to illustrate, not to limit, the disclosed embodiments.

### Example 1. N-Terminal ApoB Fragment Inhibition of Chylomicron and Low-Density Lipoprotein (LDL) Uptake by Endothelial Cells

The N-terminal region of ApoB is common to ApoB48 and ApoB100 (see, e.g., Human ApoB100 GenBank Accession No. P04114; SEQ ID NO: 1), and contains regions that could affect its interaction with proteoglycans and mediate its association with scavenger receptor-B1 (SR-B1). A second receptor, activin-like kinase 1 (ALK1) (Kraehling et al., 2016) as well as caveolin 1 may also be involved (Ramirez et al., 2019). Because association of low-density lipoprotein (LDL) with ALK1 is not competed by the low-density lipoprotein receptor (LDLR), this interaction also likely involves the N-terminal region of ApoB. SR-B1 also functions as the major receptor for high-density lipoprotein (HDL) where it participates in selective uptake of lipid without internalization of ApoA-119. The present disclosure shows that a polypeptide from the N-terminal region of ApoB blocks chylomicron and LDL uptake by ECs. Below-described experiments define characteristics that allow TG-rich lipoprotein (TRL) to bind to SR-B1 and not ALK1 and extend in vitro data to in vivo studies.

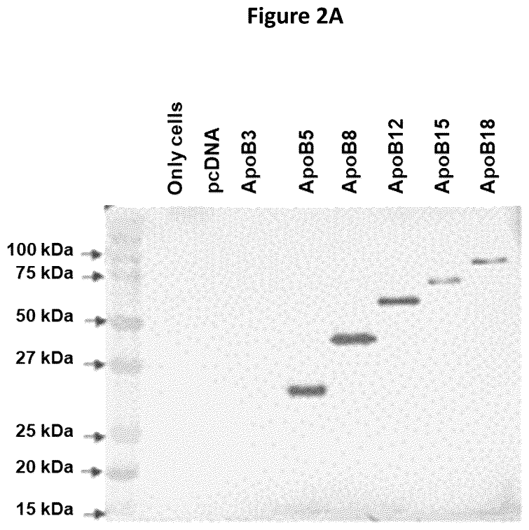

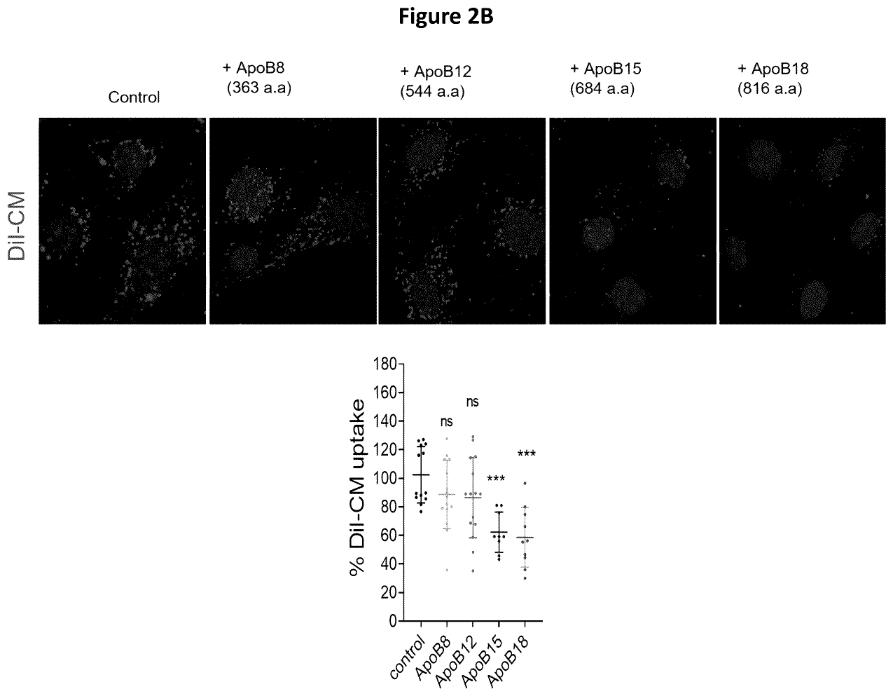

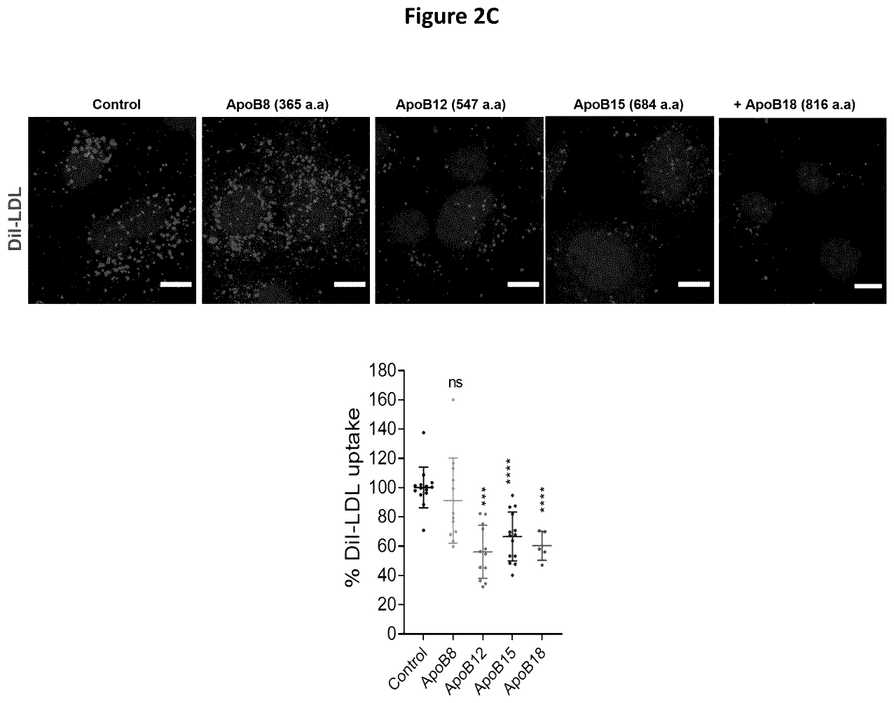

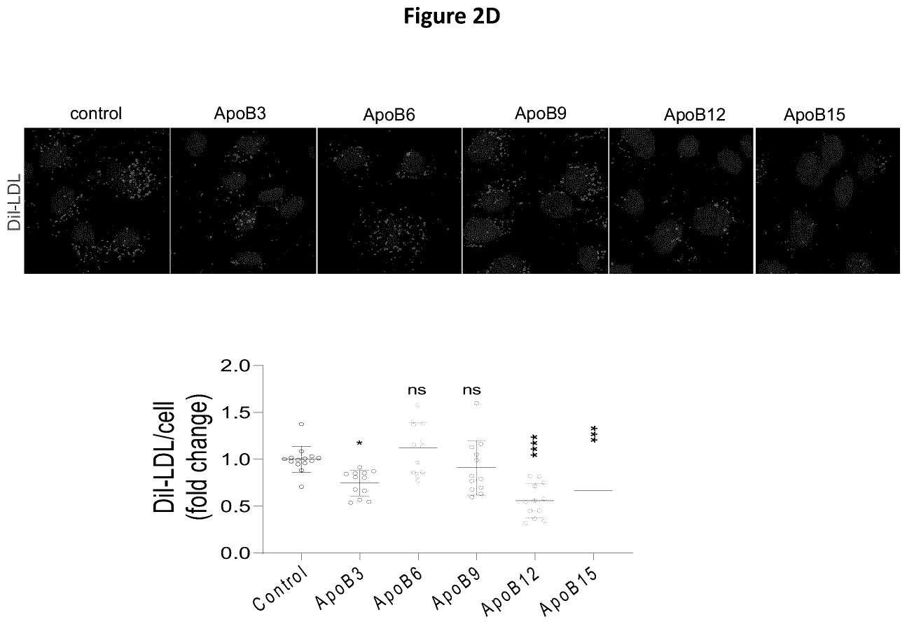

Experiments described in the present Example were designed to define the regions of ApoB required for binding to SR-B1 and ALK1. Detection of flag-tagged carboxyl-truncated ApoB polypeptides (e.g., ApoB3, ApoB5, ApoB15, and ApoB18) via agarose gel electrophoresis is depicted in FIG. 2A. Mouse endothelial cells were deprived of serum overnight, and then incubated for 30 minutes with 1,1′-Dioctadecyl 3,3,3′,3′-Tetramethylindocarbocyanine Perchlorate (DiI; red fluorescent)-labeled chylomicrons (4 mg/ml TG) or LDL (2.5 mg/dl) in the absence (control) or presence of 0.4 mg/dL ApoB polypeptides. The results showed that ApoB fragments reduce chylomicron uptake (FIG. 2A). In particular, the inhibition of chylomicron uptake required a fragment that was longer than ApoB12. Additionally, ApoB12 blocks endothelial cell DiI-LDL uptake (FIG. 2B). Hence, the ALK1 binding site was identified as between ApoB8 and ApoB12.

The present Example demonstrates polypeptides shorter than ApoB18 block either chylomicron and/or LDL uptake by endothelial cells. As shown in FIG. 2A and FIG. 2B, the region required to block LDL uptake, and likely ALK1, requires an N-terminal ApoB fragment comprising at least ApoB12 whereas inhibition of chylomicron uptake may require a longer fragment, at least ApoB15. Thus, the two receptors (i.e., SR-B1 and ALK1) interact with different regions of ApoB.

Greater postprandial lipemia has been evoked as a cause of EC dysfunction (Gaenzer et al., 2001). However, how the lipids directly affect arteries is unclear. Microscopic studies from decades ago (Parker et al., 1970) and recent studies using tracers (He et al., 2018) failed to show chylomicrons within microvascular ECs. In contrast, aortic ECs readily show lipid droplets when mouse aortas are harvested during postprandial lipemia (Kuo et al., 2017). The present inventors has shown that these ApoB-containing droplets are rapidly lost, indicating that the time required for tissue preparation is sufficient for their transcytosis or metabolism by ECs (Cabodevilla et al., 2021). Most aortic ECs do not express Glycosylphosphatidylinositol Anchored High Density Lipoprotein Binding Protein 1 (GPIHBP1), which is required for association of LpL to the luminal EC surface (Young et al., 2019). Moreover, studies by the present inventors (Yla-Herttuala et al., 1991) and others (O'Brien et al., 1992) showed that most LpL in arteries is adjacent to macrophage-rich areas within plaques. Mice with LpL deficiency have a marked increase in aortic lipid droplets, and this is associated with intracellular ApoB, indicating chylomicron uptake. This uptake appears to require SR-B1 and is inhibited by a polypeptide containing 18% ApoB from its N-terminal region. Using additional ApoB fragments, the competing region appears to be between ApoB12 and ApoB15 (FIG. 2B).

The data presented herein suggest that the ApoB-binding site in LDL is between ApoB8 and B12 (FIG. 2C and FIG. 2D). The in vivo differences in chylomicron and LDL interaction with these receptors might be due to ApoB conformations that is affected by the size of the lipoproteins, additional protein cargo due to the site of synthesis, or the length of ApoB.

Chylomicrons and LDL are both internalized by ECs, but their uptake and intracellular metabolism differs, as chylomicrons are targeted to lysosomes whereas LDL is transcytosed. Both are ligands for SR-B1. The exact sequence of ApoB required for binding to SR-B1 has not been identified. The N-terminus of ApoB likely harbors a binding sequence for SR-B1. Using C-terminally truncated fragments of human ApoB, the present Example confirms that ApoB18 and ApoB15, but not ApoB12 or smaller fragments, block chylomicron uptake into ECs (FIG. 2B). ApoB18 also reduced DiI-LDL uptake by aortic ECs. ALK1 uptake is not competed by overexpression of the LDLR, suggesting that LDL/ALK1 interaction is not via the LDL-binding region of ApoB and instead is likely via the N-terminal region (O'Brien et al., 1992). Furthermore, an antibody to this region decreases uptake of LDL via ALK1. A small fragment of ApoB that blocks LDL uptake via ALK1 did not block chylomicron uptake by ECs; this fragment was taken from a charged region between ApoB8 and ApoB12. Thus, two separate N-terminal sites in ApoB interact with EC receptors SR-B1 and ALK1. LDL, but not chylomicrons, appears to interact with both receptors.

It is unclear what would allow this differential receptor interaction between chylomicrons and LDL. and whether a similar difference affect binding and uptake of chylomicrons and VLDL. The size of the TG-rich lipoproteins (TRLs), the length of ApoB, and/or other components of the TRLs associated with their secretion from enterocytes or hepatocytes affect interaction with ALK1 and SR-B1. Because models suggest that the conformation of N-terminal ApoB100 is affected by its C-terminal region crossing over and positioning near the N-terminus, as suggested (Flood et al., 2002), it is possible that ApoB length affects EC interaction. In particular, either the size of the lipoprotein or the size of ApoB may obscure the ApoB/ALK1 interaction site. It is likely that chylomicron inhibition of LDL uptake in vitro is primarily due to blocking SR-B1. However, it is possible that the large size of these particles obscures both ALK1 and SR-B1 if the two receptors are in close proximity. Because chylomicrons are not ligands for ALK1, the 3D conformation of ApoB in chylomicrons may prevents the exposure of the ALK1 binding region.

Data disclosed herein support that the length of ApoB affects its binding to ALK1, with ApoB100 altering the conformation of a region between ApoB8 and B12. Thus, lipoproteins regardless of the site of origin will bind to both receptors. The size of the particles or other components due to enterocyte or liver production may also affect binding.

### Example 2. Anti-Atheroenic Effects of ApoB18 In Vivo

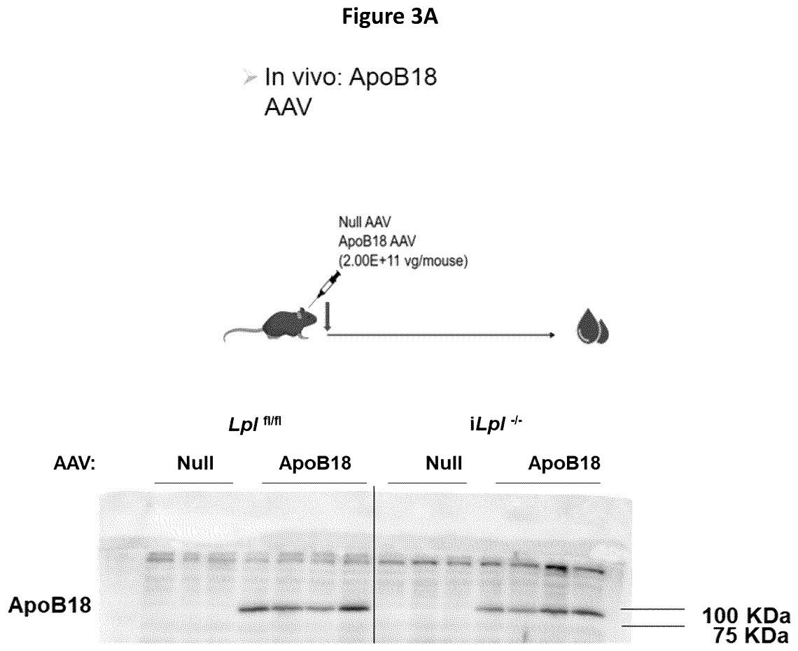

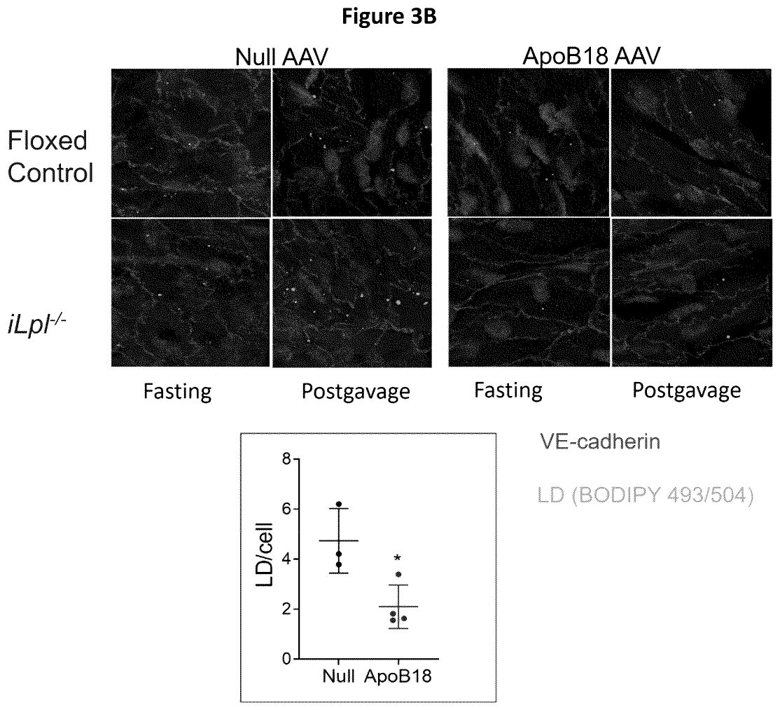

Experiments disclosed in the present Example were designed to test the potential anti-atherogenic effects of ApoB18 in vivo using adeno-associated virus (AAV) delivery approaches. Lpl^{fl/fl}(floxed control) and hypertriglyceridemic iLpl−/− mice were treated with either control (null AAV) or ApoB18 AAV (2.00E+11 vg/mouse) in accordance with the experimental timeline as depicted in FIG. 3A (top panel). Blood was obtained 8 weeks after the injection and run on a polyacrylamide gel (10%). ApoB and its fragments were identified by western blot. Plasma of mice treated with ApoB18 AAV contained high levels of circulating ApoB18. ApoB18 peptide was expressed at levels at least equal to that of full-length ApoB48 in LpL deficient mice. Thus, levels of ApoB18 were similar to those of circulating ApoB48 and ApoB100. These data demonstrate that ApoB 18 is expressed in mice after AAV injection (FIG. 3A, bottom panel) Mice were sacrificed 180 minutes after an olive oil gavage and their aortas were stained with EC marker VE-cadherin and BODIPY to assess postprandial lipid droplet accumulation, a marker for chylomicron uptake. ApoB18 AAV reduced the accumulation of chylomicron-derived postprandial lipid droplets in mouse aortas, evidence that it blocks SR-B1 in vivo (FIG. 3B). Together, data using this AAV shows high level expression, and reduced lipid droplets in the aortic of LpL deficient mice but no change in circulating lipids either fasting or in the postprandial period assessing up to 270 min. Thus, aortic uptake of chylomicrons is a minor contributor to overall lipid metabolism.

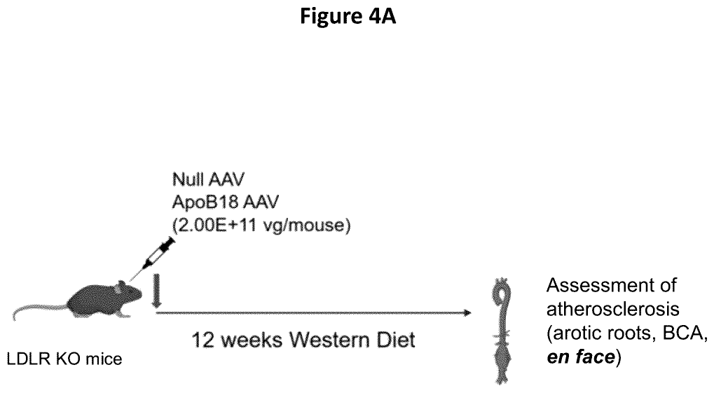

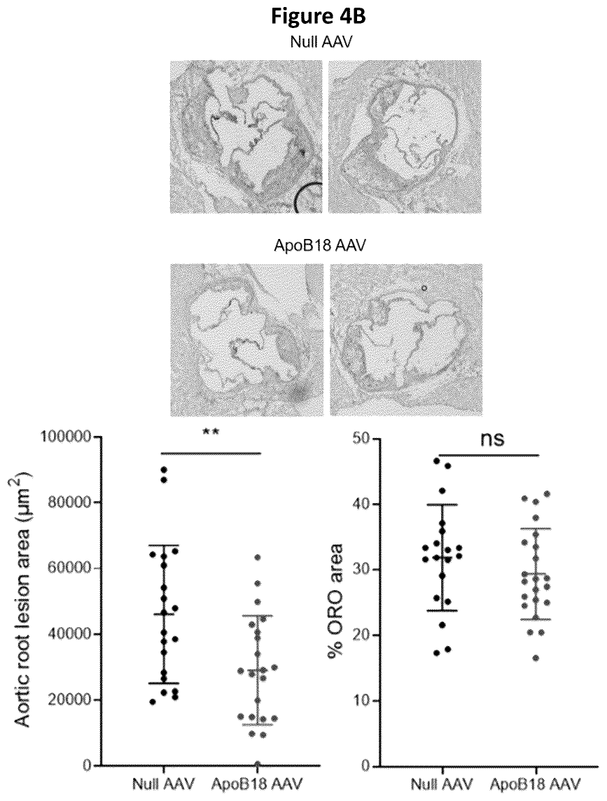

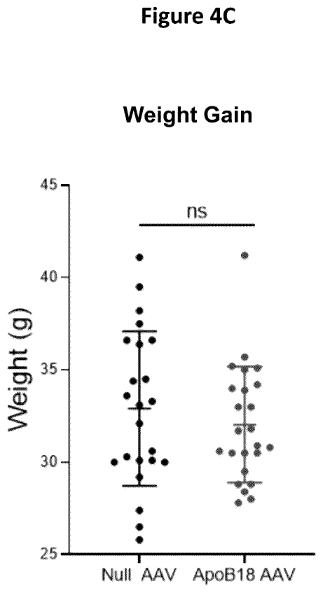

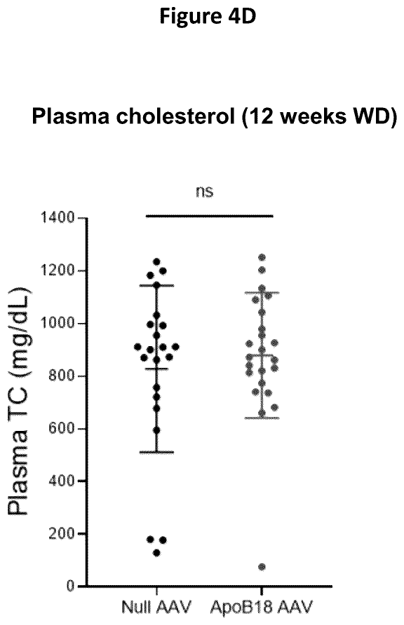

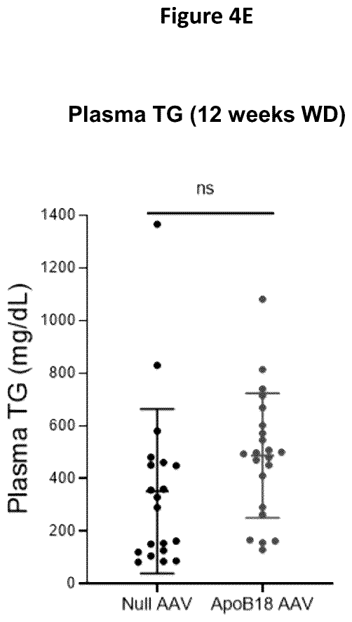

Whether ApoB18 treatment with ApoB18 AAV would reduce atherosclerosis was investigated next. Eight (8)-week-old hypercholesterolemic LDL receptor (LDLR) knock out (KO) mice (LDLR KO mice) were injected with either control (null AAV) or ApoB18 AAV and fed a western diet (WD), enriched in cholesterol and fat, for 12 weeks to induce atherosclerosis. After 12 weeks, mice were sacrificed and their aortic roots were preserved and sectioned for assessment of atherosclerotic lesion size and lipid content (FIG. 4A). Aortic roots were stained with hematoxylin/eosin for evaluation of atherosclerotic lesions (i.e., aortic root lesions) and then with Oil Red O (ORO) to stain the neutral lipid (cholesterol) (FIG. 4B, top panel). Mice treated with ApoB18 AAV exhibited significantly smaller aortic root lesions (FIG. 4B, bottom panel, left), and there was a trend towards reduction in lesion lipid content as quantified by Oil Red O (ORO) staining percentage (FIG. 4B, bottom panel, right). There was no apparent difference in weight gain (g) (FIG. 4C), plasma cholesterol (TC, total cholesterol) (mg/dL) (FIG. 4D), or plasma triglyceride (TG) (mg/dL TG) (FIG. 4E) levels between mice treated with null AAV versus ApoB18 AAV. The data from these experiments showed that ApoB18 AAV significantly reduced aortic lesion area after 12 weeks of western diet (WD) but did not profoundly alter percent lesion lipid content as monitored by ORO staining. Plasma cholesterol, triglyceride, and weight was identical regardless of treatment after WD and at time of sacrifice

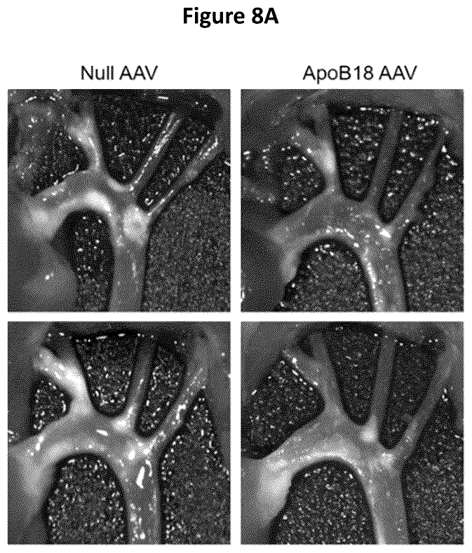

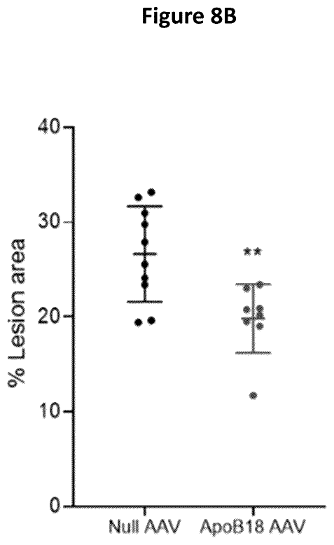

At the conclusion of the present study and prior to harvesting, the aortic artic arch of LDLR KO mice injected with either control (null AAV) or ApoB18 AAV was exposed and photographed to reveal atherosclerotic plaque (FIG. 8A, white within the images is due to atherosclerotic plaque). The images were analyzed by quantifying the percent of the aortic arch covered with white plaque and a summary of the data FIG. 8B. The data show that the percent lesion area was reduced in the ApoB18 AAV treatment group as compared to the control group (null AAV).

The present studies were performed with ApoB18, however shorter polypeptides and other small molecules can inhibit the binding of LDL to SR-B1. In some embodiments, various fragments disclosed herein may span the ligand binding site of both receptors. The precise regions of receptor interaction may be determined, and polypeptides and/or small molecules created to block both receptors.

### Example 3. ApoB100 Chylomicron Uptake is Blocked by ALK1 Knockdown

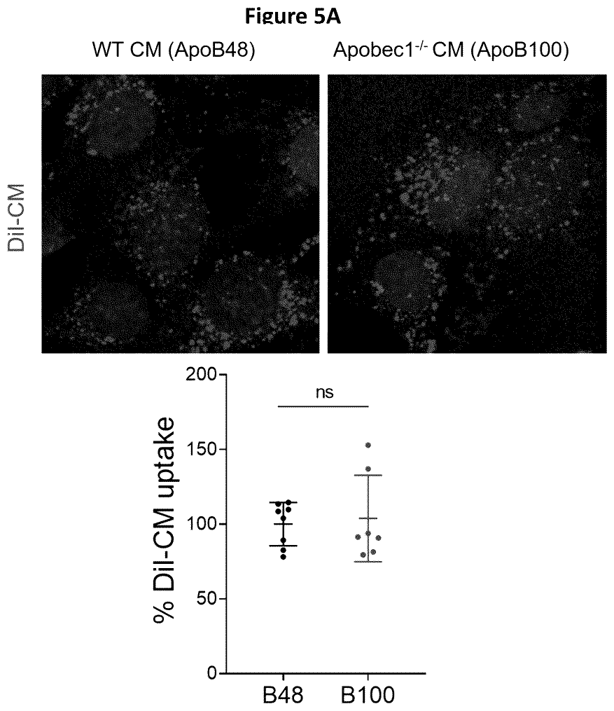

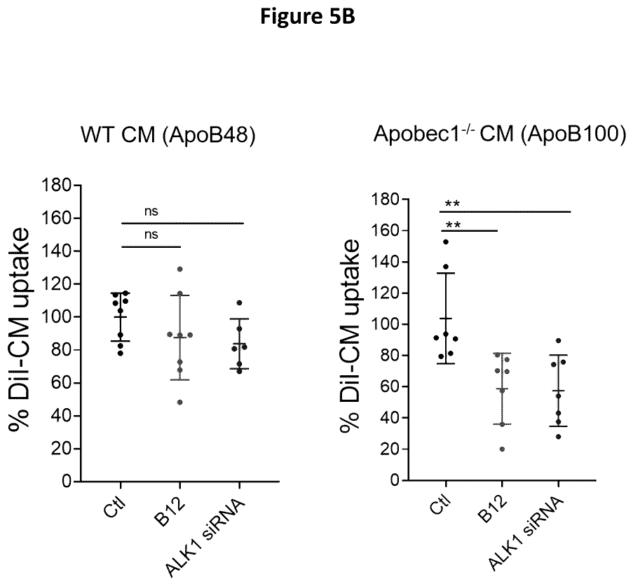

The present Example aimed to address why chylomicrons that contain a shortened fragment of ApoB, ApoB48, are internalized by SR-B1, whereas LDL that contains the longer ApoB, ApoB100, interacts with both receptors. The experiments were designed to test whether ApoB100 may expose a binding site between ApoB8 and ApoB12 that is masked in ApoB48. After a fat meal, blood was obtained from mice that only express ApoB100, and thus create ApoB100 chylomicrons. Assessment of uptake via SR-B1 and ALK1 revealed ApoB100 chylomicron uptake was blocked by ALK1 knockdown. Specifically, endothelial cells were deprived of serum overnight and exposed to WT (ApoB48-carrying) or ApoBec1⁻/− (ApoB100-carrying) chylomicrons (4 mg/dL TG) for 30 minutes. Results indicated there was no significant difference in their uptake by ECs (FIG. 5A). Inhibition of ALK1 with siRNA, and co-incubation with ApoB12 polypeptide inhibited endothelial uptake of ApoBec⁻/− but not WT chylomicrons (FIG. 5B).

### Example 4. Treatment with Chylomicrons Induces Transcription of Pro-Inflammatory Genes in ECs

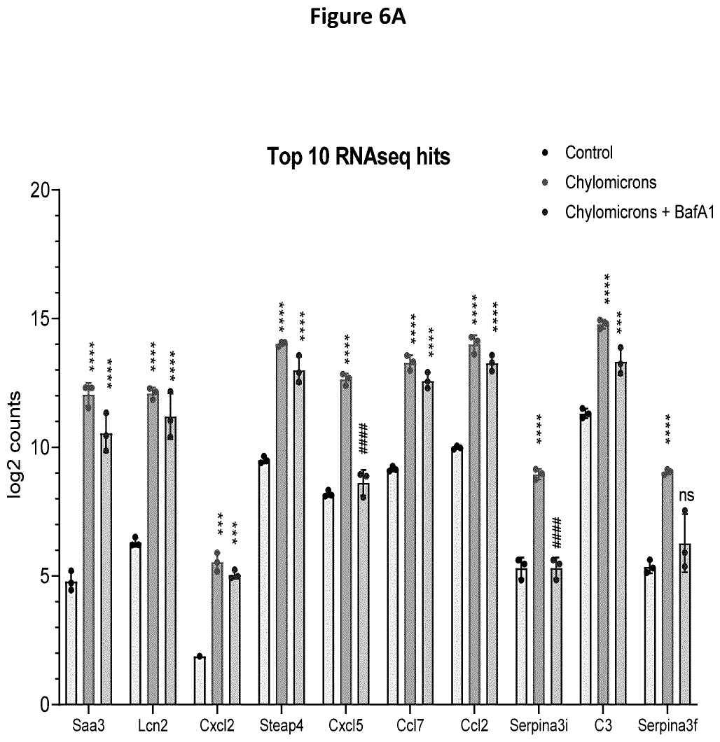

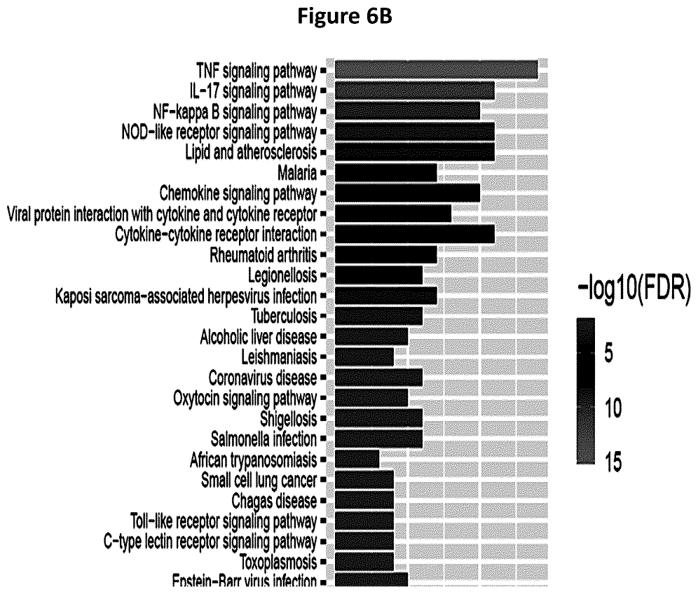

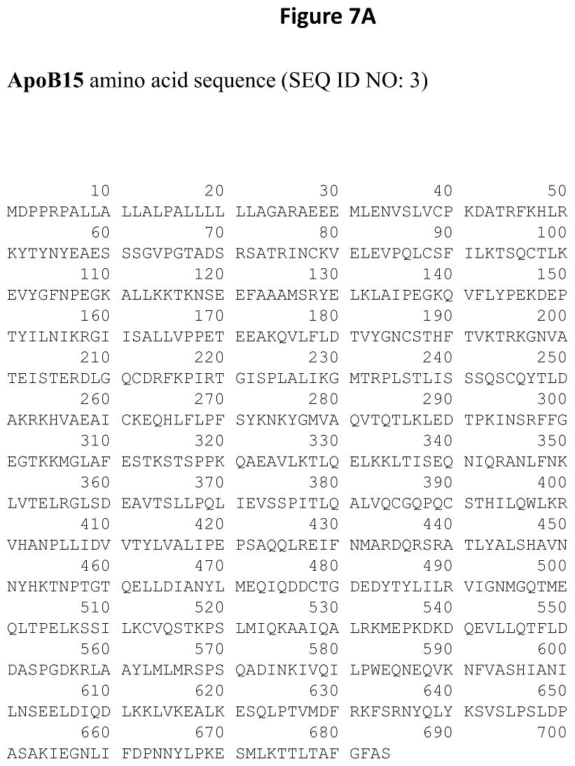

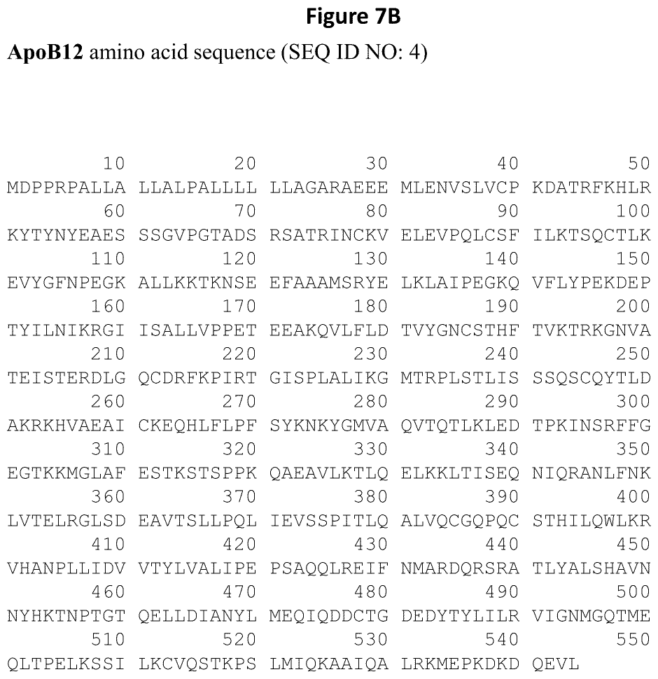

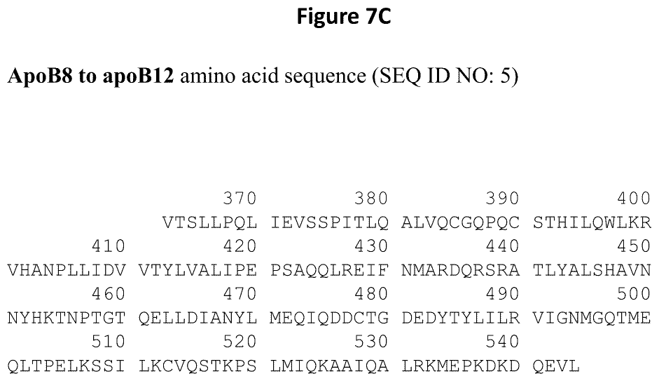

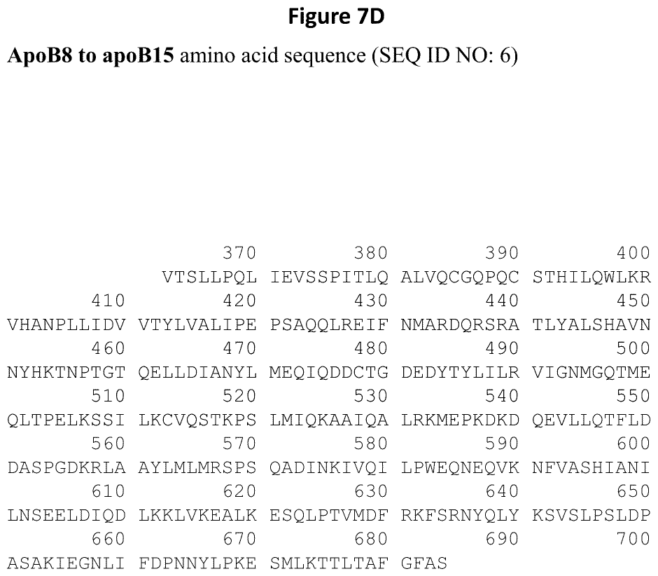

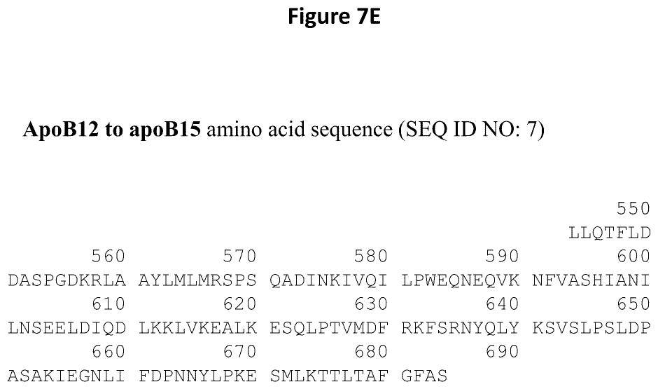

The present Example aimed to address whether endothelial cell uptake of chylomicrons affects vascular biology. As a first approach, how chylomicron uptake affects EC gene expression was assessed using RNA sequencing (RNA-seq) approaches. RNA-seq analysis of control and chylomicron-treated cells was performed using conventional methodology known in the art. As shown in FIG. 6A, the top 10 transcripts induced by this treatment were inflammatory markers. Also conventional markers associated with greater atherosclerosis, e.g., vascular cell adhesion molecule 1 (VCAM1) and intercellular adhesion molecule (ICAM), were upregulated. A heatmap displaying transcriptome profile categorization for the inflammatory genes from the RNA-Seq dataset is shown in FIG. 6B. Together, these data provide supporting evidence that blocking EC uptake by aortic ECs is anti-inflammatory and reduces atherosclerosis.

### Example 5. ApoB18 Competition and Endothelial Cell SR-B1 Knockout Reduce Endothelial Cell Inflammation

The present Example investigates reduced inflammation due to blocking chylomicron/SR-B1 pathways. A fragment of ApoB (ApoB18) protein reduced chylomicron and LDL uptake into ECs (see, e.g., FIG. 2). Moreover, ApoB18 also reduced LDL and DiI-LDL uptake by aortic ECs. Here, whether EC-specific deletion of SR-B1 or ApoB18 overexpression alters EC gene expression as well as lipid accumulation is tested. An ApoB18-expressing AAV is used to “flood” the circulation of control, and iLp⁻/− mice with ApoB18. Changes in fasting and postprandial lipoproteins and delivery of their lipid cargo to ECs and parenchymal cells is specifically assessed. Data using the ApoB18-expressing AAV show high level expression, and reduced lipid droplets in the aortic of LpL deficient mice (see, e.g., FIG. 3A and FIG. 3B) but no change in circulating lipids either fasting or in the postprandial period assessing up to 270 min. Thus, aortic uptake of chylomicrons is a minor contributor to overall lipid metabolism.

The virus (e.g., AAV8, 10¹² MOI [multiplicity of infection]) is injected intravenously, and levels of ApoB18 in the serum are monitored by western blotting. Both overnight and 4-hour fasting lipids are determined after 1 month of viral injection. A weekly gavage of olive oil spiked with retinol is performed to assess postprandial lipemia of chylomicrons and remnants. Similar studies are performed in genetic knockout mice missing LpL and EC SR-B1. These analyses are performed in, e.g., 10 mice (half males and half females), allowing each animal to serve as its own control. Five mice of each genotype receive non-producing AAV to serve as another control.

To determine if ApoB18 affects lipid uptake into ECs, lipid droplets are assessed in ECs when the 1-month studies are completed. The experimental focus is on accumulation of droplets at 3 hours after a gavage, a time when droplet formation usually peaks in wild type and iLpl-mice. Accumulation of aortic lipid droplets is assessed. Specifically, the mouse aorta is rapidly removed, washed with 4° C. PBS, and stained with BODIPY (for lipids) and VE-Cadherin. The aortas are assessed by confocal microscopy, and VE-Cadherin staining is used to confirm the level of the aortic ECs. The atherosclerosis-prone lesions are identified by co-immunofluorescence staining of CD31 and either ICAM1 or VCAM1. The relative numbers of lipid droplets and their sizes are determined. Cells are stained for ApoB48, ApoB100, and perilipin 2 to determine whether the droplets represent internalized non-digested lipoproteins or are perilipin-containing lipid droplets assembled within cells. The lipid droplets are stained with BODIPY and VE-cadherin with antibodies to ensure that the confocal imaging is of the ECs. ECs are isolated using conventional methodology by those skilled in the art. Gene analysis is assessed by RNA-seq and corresponding pathway analysis is performed.

ApoB18 is overexpressed and high levels of the ApoB18 polypeptide are produced in the blood. ApoB18 production does not depend on microsomal triglyceride transfer protein (MTP), and ApoB18 has been overexpressed and secreted by cultured cells (Cover et al., 2033; Bakillah et al., 1998). Next, the smallest polypeptide and thus the region(s) in ApoB that mediates the ApoB18 effect is defined. A number of ApoB polypeptides, including but not limited to those comprising various mutations, are generated. First, several charged regions are mutated and whether the mutated proteins affect binding to SR-B1 is determined in vitro, followed by chylomicron uptake by cells, before using the mutated polypeptides in vivo. Initial studies are performed using human ApoB18. ApoB is highly (81.9%) homologous between human and mouse; the NH₂-terminal region is 88.8% homologous (Johs et al., 2006). Findings are repeated using an AAV that expresses mouse ApoB18.

ApoB18 reduces chylomicron uptake into aortic ECs via blocking both ALK1 and SR-B1. Changes in EC lipids and gene expression in LpL knockout mice and during the postprandial period are identified.

### Example 6. Effects of N-Terminal ApoB Fragments on Atherosclerosis

The present Example is designed to investigate ApoB15-versus ApoB12-associated reduction in chylomicron-induced atherosclerosis. As shown in FIG. 2, selective uptake of chylomicrons versus LDL is inhibited by using different length N-terminal ApoB fragments. Here, the importance of SR-B1 and ALK1 in atherogenesis is established, and whether blocking added vascular inflammation due to chylomicrons will neutralize the greater lesion size in mice with a combined knockout of LpL and LDLR is determined.

The iLp^{1/-} mice are crossed with LDLR knockout mice. Whether LpL deficiency and hyperTG increase atherosclerosis after a 12-week Western Diet is assessed. Both ApoB12 and ApoB15 AAVs are generated using standard techniques known in the art, there in vivo expression, as well as lack of changes in circulating lipoprotein levels is confirmed. The study will replicate the conditions, e.g., such as those used in Example 2 (above), but with inclusion of the AAV8 in half the mice beginning at 6 weeks when the Western diet is initiated.

Atherosclerosis is assessed in the aortic root, brachiocephalic artery (BCA), and entire aorta. Images of the aortic arch with direct visualization are obtained (Vikramadithyan et al., 2005). The percent of the block occupied by macrophages, collagen, and lipid are quantified and the amount of necrotic core determined. Lesions are assessed for whole vessel transcriptomics using standard methods known to those of skill in the art to separate expression patterns in ECs, macrophages, vascular smooth muscle cells (VSMCs), and other plaque cells.

N-terminal fragments that block interaction of chylomicrons and also LDL with EC receptors reduce atherosclerosis. Studies akin to those of others (Ramirez et al., 2019; O'Brien et al., 1992) are performed to assess lipoprotein transcytosis. Both ALK1 and SR-B1 are needed for this process, and ApoB12 and ApoB15 can be equally effective in non-hyperTG mice. ApoB15 alone may reduce both LDL and chylomicron driven atherosclerosis, i.e., ApoB18-treated mice can have less atherosclerosis than ApoB15-treated mice, consistent with studies of EC inflammation disclosed herein.

### Example 7. Determination of the Region of ApoB18 that Mediates its Interaction with SR-B1

The present Example investigates the N-terminal region of ApoB, which contains two distinct sites whose exposure and metabolism via ALK1 and SR-B1 leads to differences in EC uptake and metabolism, and small extracellular vesicle (sEV) production. The smallest N-terminal ApoB peptide that effectively binds SR-B1 is determined and then charged residues in that sequence are mutated to pinpoint the specific binding domain(s). In addition, the differential binding of ApoB to SR-B1 versus ALK1 is examined. EC SR-B1 is knocked down using antisense oligonucleotides (ASOs) as described, for example, in Cabodevilla et al., 2021, and siRNA is developed to knock down ALK1.

To determine why SR-B1 and ALK1 mediate LDL EC transcytosis, but chylomicrons only bind to SR-B1, carboxyl-terminus truncated ApoB peptides are used to confirm that the regions blocking each receptor differ. To determine whether the size of the particles or the ApoB length affect binding to ALK1, ApoB100 chylomicrons and ApoB48 LDL are created, and the binding of these lipoproteins to each receptor is assessed. A colony of APOBEC1 knockout mice, which only express ApoB100, are used. Chylomicrons are produced 3 hours after a 200 μl after a corn oil gavage, serum incubated with DiI, and short centrifugation (45 min×26,000 g) are used to isolate the large TRLs, and LDL isolated from the infranatant. Unlike humans, mouse LDL contains a high percent of ApoB48; LDL from LDLR knockout is 30-50% ApoB48. Both an ASO and proprotein convertase subtilisin/kexin type 9 (PCSK9) AAV are used to create LDLR knockdown, and this is used to generate pure ApoB100 LDL in the APOBEC1 knockout mice. Mice expressing only ApoB48 are available from Jackson Lab. These mice are obtained to create pure ApoB48 LDL and, as a control, pure ApoB48 chylomicrons. As a corollary to these studies, mix studies using mouse and human particles and cells are performed. Human LDL is almost entirely ApoB100 and human chylomicrons (e.g., those the inventors of the disclosure can harvest from LpL deficient patients) is primarily ApoB48. Whether mouse and human cells handle the mouse and human particles similarly is determined.

Two lysine-rich regions found between ApoB15 and ApoB18 have been proposed to mediate ApoB interaction with scavenger receptors (Kreuzer et al., 1997); these are at residues 612-620. ApoB18 is mutated by changing lysines in each of these regions to alanines, and thus the importance of each of these sites is determined. Polypeptides are created from that region containing each site to use as competitive inhibitors of chylomicron and LDL SR-B1 uptake and then these sites are mutated in ApoB48 and ApoB100 (which are in lipoproteins).

The regions of ApoB required for uptake via SR-B1 and ALK1 differ. Additional experiments more precisely define the ApoB ligand region for SR-B1 by first using smaller polypeptides and then mutagenesis. Failure of chylomicrons to interact with ALK1 can be due to a conformational change in ApoB. Whether this is due to the size of the particle or differences in the exposure of the ALK1 binding site is evident from the experiments using lipoproteins with different ApoBs. Another approach is to mutagenize the binding regions in ApoB48 and ApoB100. Whether circulating LDL and larger lipoproteins from control and diabetic rabbits interact with SR-B1 and/or ALK1 is determined.

As an alternative approach to assess LDL and chylomicron binding to SR-B1, binding assays and immunoprecipitation of SR-B1 are performed. For example, ECs are created that express his-tagged wild type and non-binding mutants of SR-B1 in cells with CRISPR-deleted native SR-B1. Binding and crosslinking are used to detect ApoB/SR-B1 complexes or complexes with truncated or mutant ApoB fragments, as shown for SR-B1-LDL-DOCK4 complexes (Huang et al., 2019), and uncovers intracellular proteins that target the SR-B1/chylomicron complex to the lysosome. Experiments show LDL binding to SR-B1 and ALK1 involves two separate sites and can indicate that both receptors are simultaneously occupied by a single LDL.

### Example 8. Blocking Endothelial Cell (EC) Uptake of Chylomicrons Reduces Atherosclerosis

The present Example discloses various experimental paradigms to test ApoB polypeptide-associated reduction in atherosclerosis. An overall goal of the present disclosure is to define the chylomicron/EC/SR-B1 pathway and its role in atherosclerosis. Along with blocking chylomicron uptake, ApoB18 blocks LDL uptake into ECs. These in vitro studies suggest that ApoB18 blocks uptake via both SR-B1 and ALK1. The present Example, which can have translational implications, is designed to test whether AAV ApoB18 reduces atherosclerosis, followed by determination of whether this effect is primarily due to inhibition of SR-B1 or ALK1.

To determine whether ApoB18 affects atherosclerosis, Ldlr^{-/-} mice express AAV ApoB18 or a control AAV beginning at 6 weeks and then are switched to a western diet. Plasma lipids and white blood cells (WBCs) are monitored weekly, as well as apo18 expression. Atherosclerosis is determined at 10 and 16 weeks. Lesion development is altered and ApoB18 polypeptide-associated reduction of atherosclerosis in the EC-SR-B1, EC-ALK1, and/or iLpl^{-/-}/Ldlr^{-/-} models is determined. Various atherosclerosis assays are performed. Briefly, as an example, direct visualization of the whole aorta, digitalization/morphometrics of aortic root lesion area, macrophage (CD68⁺) area, collagen content, and Oil Red O-stained lipid area are used. Chylomicrons, especially those in iLpl mice that have prolonged circulation, may be enriched in oxidized phospholipids, which others have implicated in atherogenesis (Que et al., 2018). Lp(a) is also thought to be more atherogenic due to its composition of similar oxidized lipids (see, e.g., Tsimikas and Witztum, Curr Opin Lipidol. 2008 August; 19(4):369-77, PMID 18607184).

To determine effects on the necrotic core, an important determinant of plaque stability in humans, efferocytosis levels (which are directly and indirectly related, respectively, to core size), is measured, in addition to apoptosis. VSMCs and collagen content is measured by immunostaining with VSMC-specific markers (e.g., a-actin) and Sirius red staining, respectively. Cells that stain with both VSMC actin and CD68 are separately assessed, are likely to be VSMC-derived foam cells (Gomez et al., 2013; Allahverdian et al., 2014). CD68⁺ and the double-positive cells are then obtained using laser capture microdissection (LCM) (e.g., via methods as described in Trogan et al., 2002 and Rahman et al., 2017). For a targeted assessment of the inflammatory states of the macrophages and the double-positive cells from LCM samples, RNA-seq is used to produce transcriptome profiles of these populations. The inflammatory phenotype is of particular interest. For higher resolution, an expression of a panel of, e.g., M1- and M2-associated genes is specifically assayed. Brachiocephalic arteries (BCAs) are similarly studied. Lipidomic analyses are performed in plasma, arteries, ECs, and macrophages.

The ability to separate subpopulations by distinct transcriptome profiles permits differentiation of macrophages that originate from monocytes from those that originate from VSMCs (Dobnikar et al., 2018). A more extensive scRNA-seq approach is used to examine the different types of cells in plaques.

ApoB18 reduces chylomicron uptake into aortic ECs and alters LDL transcytosis by blocking both ALK1 and SR-B1. ApoB18 alters EC lipids and gene expression. This is evident in the LpL-deficient mice. Experiments disclosed herein define the determinants of LDL and chylomicron EC uptake. These data allow use of smaller blocking polypeptides. Initial studies were performed using human ApoB18. ApoB is highly (81.9%) homologous between human and mouse; the NH₂-terminal region is 88.8% homologous. Findings are recapitulated using an AAV that expresses mouse ApoB18. Additional studies are designed to interrogate atherosclerosis in mice with mutated ApoB. There are some data that implicate ApoB-mediated immune activation as affecting atherosclerosis (Tsiantoulas et al., 2014), with some antibodies appear to be protective and others harmful. Immune activation by assessing circulating antibodies to ApoB, B-cell activation, and immune complexes (ApoB18-IgG) in arteries (and kidneys) are monitored. Antibody production using a monoclonal antibody to CD20, as is done using the clinical therapy rituximab, is used to determine whether this negates the greater atherosclerosis.

### REFERENCES

Adiels M, Matikainen N, Westerbacka J, Soderlund S, Larsson T, Olofsson S O, Boren J and Taskinen M R. Postprandial accumulation of chylomicrons and chylomicron remnants is determined by the clearance capacity. Atherosclerosis. 2012; 222:222-8.
Allahverdian S, Chehroudi A C, McManus B M, Abraham T and Francis G A. Contribution of intimal smooth muscle cells to cholesterol accumulation and macrophage-like cells in human atherosclerosis. Circulation. 2014; 129:1551-9.
Bakillah A, Jamil H and Hussain M M. Lysine and arginine residues in the N-terminal 18% of apolipoprotein B are critical for its binding to microsomal triglyceride transfer protein. Biochemistry. 1998; 37:3727-34.
Cabodevilla A G, Tang S, Lee S, Mullick A E, Aleman J O, Hussain M M, Sessa W C, Abumrad N A and Goldberg I J. Eruptive xanthoma model reveals endothelial cells internalize and metabolize chylomicrons, leading to extravascular triglyceride accumulation. J Clin Invest. 2021; 131.
Covey S D, Krieger M, Wang W, Penman M and Trigatti B L. Scavenger receptor class B type I-mediated protection against atherosclerosis in LDL receptor-negative mice involves its expression in bone marrow-derived cells. Arterioscler Thromb Vasc Biol. 2003; 23:1589-94.
Dobnikar L, Taylor A L, Chappell J, Oldach P, Harman J L, Oerton E, Dzierzak E, Bennett M R, Spivakov M and Jorgensen H F. Disease-relevant transcriptional signatures identified in individual smooth muscle cells from healthy mouse vessels. Nat Commun. 2018; 9:4567.
Flood C, Gustafsson M, Richardson P E, Harvey S C, Segrest J P and Boren J. Identification of the proteoglycan binding site in apolipoprotein B48. J Biol Chem. 2002; 277:32228-33.
Gaenzer H, Sturm W, Neumayr G, Kirchmair R, Ebenbichler C, Ritsch A, Foger B, Weiss G and Patsch J R. Pronounced postprandial lipemia impairs endothelium-dependent dilation of the brachial artery in men. Cardiovasc Res. 2001; 52:509-16.
Gomez D, Shankman L S, Nguyen A T and Owens G K. Detection of histone modifications at specific gene loci in single cells in histological sections. Nat Methods. 2013; 10:171-7.
He C, Weston T A, Jung R S, Heizer P, Larsson M, Hu X, Allan C M, Tontonoz P, Reue K, Beigneux A P, Ploug M, Holme A, Kilburn M, Guagliardo P, Ford D A, Fong L G, Young S G and Jiang H. NanoSIMS Analysis of Intravascular Lipolysis and Lipid Movement across Capillaries and into Cardiomyocytes. Cell Metab. 2018; 27:1055-1066 e3.
Huang L, Chambliss K L, Gao X, Yuhanna I S, Behling-Kelly E, Bergaya S, Ahmed M, Michaely P, Luby-Phelps K, Darehshouri A, Xu L, Fisher E A, Ge W P, Mineo C and Shaul P W. S R-B1 drives endothelial cell LDL transcytosis via DOCK4 to promote atherosclerosis. Nature. 2019; 569:565-569.
Johs A, Hammel M, Waldner I, May R P, Laggner P and Prassl R. Modular structure of solubilized human apolipoprotein B-100. Low resolution model revealed by small angle neutron scattering. J Biol Chem. 2006; 281:19732-9.
Kraehling J R, Chidlow J H, Rajagopal C, Sugiyama M G, Fowler J W, Lee M Y, Zhang X, Ramirez C M, Park E J, Tao B, Chen K, Kuruvilla L, Larrivee B, Folta-Stogniew E, Ola R, Rotllan N, Zhou W, Nagle M W, Herz J, Williams K J, Eichmann A, Lee W L, Fernandez-Hernando C and Sessa W C. Genome-wide RNAi screen reveals ALK1 mediates LDL uptake and transcytosis in endothelial cells. Nature communications. 2016; 7:13516.
Kreuzer J, White A L, Knott T J, Jien M L, Mehrabian M, Scott J, Young S G and Haberland M E. Amino terminus of apolipoprotein B suffices to produce recognition of malondialdehyde-modified low density lipoprotein by the scavenger receptor of human monocyte-macrophages. J Lipid Res. 1997; 38:324-42.
Kuo A, Lee M Y and Sessa W C. Lipid Droplet Biogenesis and Function in the Endothelium. Circ Res. 2017; 120:1289-1297.
O'Brien K D, Gordon D, Deeb S, Ferguson M and Chait A. Lipoprotein lipase is synthesized by macrophage-derived foam cells in human coronary atherosclerotic plaques. J Clin Invest. 1992; 89:1544-50.
Que X, Hung M Y, Yeang C, Gonen A, Prohaska T A, Sun X, Diehl C, Maatta A, Gaddis D E, Bowden K, Pattison J, MacDonald J G, Yla-Herttuala S, Mellon P L, Hedrick C C, Ley K, Miller Y I, Glass C K, Peterson K L, Binder C J, Tsimikas S and Witztum J L. Oxidized phospholipids are proinflammatory and proatherogenic in hypercholesterolaemic mice. Nature. 2018; 558:301-306.
Rahman K, Vengrenyuk Y, Ramsey S A, Vila N R, Girgis N M, Liu J, Gusarova V, Gromada J, Weinstock A, Moore K J, Loke P and Fisher E A. Inflammatory Ly6Chi monocytes and their conversion to M2 macrophages drive atherosclerosis regression. J Clin Invest. 2017; 127:2904-2915.
Ramirez C M, Zhang X, Bandyopadhyay C, Rotllan N, Sugiyama M G, Aryal B, Liu X, He S, Kraehling J R, Ulrich V, Lin C S, Velazquez H, Lasuncion M A, Li G, Suarez Y, Tellides G, Swirski F K, Lee W L, Schwartz M A, Sessa W C and Fernandez-Hernando C. Caveolin-1 Regulates Atherogenesis by Attenuating Low-Density Lipoprotein Transcytosis and Vascular Inflammation
Independently of Endothelial Nitric Oxide Synthase Activation. Circulation. 2019; 140:225-239. Swirski F K, Lee W L, Schwartz M A, Sessa W C and Fernandez-Hernando C. Caveolin-1 Regulates Atherogenesis by Attenuating Low-Density Lipoprotein Transcytosis and Vascular Inflammation Independently of Endothelial Nitric Oxide Synthase Activation. Circulation. 2019; 140:225-239.
Trogan E, Choudhury R P, Dansky H M, Rong J X, Breslow J L and Fisher E A. Laser capture microdissection analysis of gene expression in macrophages from atherosclerotic lesions of apolipoprotein E-deficient mice. Proc Natl Acad Sci USA. 2002; 99:2234-9.
Tsiantoulas D, Diehl C J, Witztum J L and Binder C J. B cells and humoral immunity in atherosclerosis. Circ Res. 2014; 114:1743-56.
Tsimikas and Witztum, The role of oxidized phospholipids in mediating lipoprotein(a) atherogenicity. Curr Opin Lipidol. 2008 August; 19(4):369-77.
Vaisman B L, Vishnyakova T G, Freeman L A, Amar M J, Demosky S J, Liu C, Stonik J A, Sampson M L, Pryor M, Bocharov A V, Eggerman T L, Patterson A P and Remaley A T. Endothelial Expression of Scavenger Receptor Class B, Type I Protects against Development of Atherosclerosis in Mice. Biomed Res Int. 2015; 2015:607120.
Vikramadithyan R K, Hu Y, Noh H L, Liang C P, Hallam K, Tall A R, Ramasamy R and Goldberg I J. Human aldose reductase expression accelerates diabetic atherosclerosis in transgenic mice. J Clin Invest. 2005; 115:2434-43.
Yla-Herttuala S, Lipton B A, Rosenfeld M E, Goldberg I J, Steinberg D and Witztum J L. Macrophages and smooth muscle cells express lipoprotein lipase in human and rabbit atherosclerotic lesions. Proc Natl Acad Sci USA. 1991; 88:10143-7.
Young S G, Fong L G, Beigneux A P, Allan C M, He C, Jiang H, Nakajima K, Meiyappan M, Birrane G and Ploug M. GPIHBP1 and Lipoprotein Lipase, Partners in Plasma Triglyceride Metabolism. Cell Metab. 2019; 30:51-65.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

List of Sequences

SEQ ID NO: 1 ApoB100 (ApoB100) amino acid sequence
MDPPRPALLALLALPALLLLLLAGARAEEEMLENVSLVCPKDATRFKHLRKYTYNYEA
ESSSGVPGTADSRSATRINCKVELEVPQLCSFILKTSQCTLKEVYGFNPEGKALLKKTKN
SEEFAAAMSRYELKLAIPEGKQVFLYPEKDEPTYILNIKRGIISALLVPPETEEAKQVLFLD
TVYGNCSTHFTVKTRKGNVATEISTERDLGQCDRFKPIRTGISPLALIKGMTRPLSTLISSS
QSCQYTLDAKRKHVAEAICKEQHLFLPFSYKNKYGMVAQVTQTLKLEDTPKINSRFFGE
GTKKMGLAFESTKSTSPPKQAEAVLKTLQELKKLTISEQNIQRANLFNKLVTELRGLSDE
AVTSLLPQLIEVSSPITLQALVQCGQPQCSTHILQWLKRVHANPLLIDVVTYLVALIPEPS
AQQLREIFNMARDQRSRATLYALSHAVNNYHKTNPTGTQELLDIANYLMEQIQDDCTG
DEDYTYLILRVIGNMGQTMEQLTPELKSSILKCVQSTKPSLMIQKAAIQALRKMEPKDK
DQEVLLQTFLDDASPGDKRLAAYLMLMRSPSQADINKIVQILPWEQNEQVKNFVASHIA
NILNSEELDIQDLKKLVKEALKESQLPTVMDFRKFSRNYQLYKSVSLPSLDPASAKIEGN
LIFDPNNYLPKESMLKTTLTAFGFASADLIEIGLEGKGFEPTLEALFGKQGFFPDSVNKAL
YWVNGQVPDGVSKVLVDHFGYTKDDKHEQDMVNGIMLSVEKLIKDLKSKEVPEARAY
LRILGEELGFASLHDLQLLGKLLLMGARTLQGIPQMIGEVIRKGSKNDFFLHYIFMENAF
ELPTGAGLQLQISSSGVIAPGAKAGVKLEVANMQAELVAKPSVSVEFVTNMGIIIPDFAR
SGVQMNTNFFHESGLEAHVALKAGKLKFIIPSPKRPVKLLSGGNTLHLVSTTKTEVIPPLI
ENRQSWSVCKQVFPGLNYCTSGAYSNASSTDSASYYPLTGDTRLELELRPTGEIEQYSVS
ATYELQREDRALVDTLKFVTQAEGAKQTEATMTFKYNRQSMTLSSEVQIPDFDVDLGTI
LRVNDESTEGKTSYRLTLDIQNKKITEVALMGHLSCDTKEERKIKGVISIPRLQAEARSEI
LAHWSPAKLLLQMDSSATAYGSTVSKRVAWHYDEEKIEFEWNTGTNVDTKKMTSNFP
VDLSDYPKSLHMYANRLLDHRVPQTDMTFRHVGSKLIVAMSSWLQKASGSLPYTQTLQ
DHLNSLKEFNLQNMGLPDFHIPENLFLKSDGRVKYTLNKNSLKIEIPLPFGGKSSRDLKM
LETVRTPALHFKSVGFHLPSREFQVPTFTIPKLYQLQVPLLGVLDLSTNVYSNLYNWSAS
YSGGNTSTDHFSLRARYHMKADSVVDLLSYNVQGSGETTYDHKNTFTLSYDGSLRHKF
LDSNIKFSHVEKLGNNPVSKGLLIFDASSSWGPQMSASVHLDSKKKQHLFVKEVKIDGQ
FRVSSFYAKGTYGLSCQRDPNTGRLNGESNLRFNSSYLQGTNQITGRYEDGTLSLTSTSD
LQSGIIKNTASLKYENYELTLKSDTNGKYKNFATSNKMDMTFSKQNALLRSEYQADYES
LRFFSLLSGSLNSHGLELNADILGTDKINSGAHKATLRIGQDGISTSATTNLKCSLLVLEN
ELNAELGLSGASMKLTTNGRFREHNAKFSLDGKAALTELSLGSAYQAMILGVDSKNIFN
FKVSQEGLKLSNDMMGSYAEMKFDHTNSLNIAGLSLDFSSKLDNIYSSDKFYKQTVNLQ
LQPYSLVTTLNSDLKYNALDLTNNGKLRLEPLKLHVAGNLKGAYQNNEIKHIYAISSAA
LSASYKADTVAKVQGVEFSHRLNTDIAGLASAIDMSTNYNSDSLHFSNVFRSVMAPFTM
TIDAHTNGNGKLALWGEHTGQLYSKFLLKAEPLAFTFSHDYKGSTSHHLVSRKSISAAL
EHKVSALLTPAEQTGTWKLKTQFNNNEYSQDLDAYNTKDKIGVELTGRTLADLTLLDS
PIKVPLLLSEPINIIDALEMRDAVEKPQEFTIVAFVKYDKNQDVHSINLPFFETLQEYFERN
RQTIIVVLENVQRNLKHINIDQFVRKYRAALGKLPQQANDYLNSFNWERQVSHAKEKLT
ALTKKYRITENDIQIALDDAKINFNEKLSQLQTYMIQFDQYIKDSYDLHDLKIAIANIIDEII
EKLKSLDEHYHIRVNLVKTIHDLHLFIENIDENKSGSSTASWIQNVDTKYQIRIQIQEKLQ
QLKRHIQNIDIQHLAGKLKQHIEAIDVRVLLDQLGTTISFERINDILEHVKHFVINLIGDFE
VAEKINAFRAKVHELIERYEVDQQIQVLMDKLVELAHQYKLKETIQKLSNVLQQVKIKD
YFEKLVGFIDDAVKKLNELSFKTFIEDVNKFLDMLIKKLKSFDYHQFVDETNDKIREVTQ
RLNGEIQALELPQKAEALKLFLEETKATVAVYLESLQDTKITLIINWLQEALSSASLAHM
KAKFRETLEDTRDRMYQMDIQQELQRYLSLVGQVYSTLVTYISDWWTLAAKNLTDFAE
QYSIQDWAKRMKALVEQGFTVPEIKTILGTMPAFEVSLQALQKATFQTPDFIVPLTDLRI
PSVQINFKDLKNIKIPSRFSTPEFTILNTFHIPSFTIDFVEMKVKIIRTIDQMLNSELQWPVP
DIYLRDLKVEDIPLARITLPDFRLPEIAIPEFIIPTLNLNDFQVPDLHIPEFQLPHISHTIEVPT
FGKLYSILKIQSPLFTLDANADIGNGTTSANEAGIAASITAKGESKLEVLNFDFQANAQLS
NPKINPLALKESVKFSSKYLRTEHGSEMLFFGNAIEGKSNTVASLHTEKNTLELSNGVIV
KINNQLTLDSNTKYFHKLNIPKLDFSSQADLRNEIKTLLKAGHIAWTSSGKGSWKWACP
RFSDEGTHESQISFTIEGPLTSFGLSNKINSKHLRVNQNLVYESGSLNFSKLEIQSQVDSQH
VGHSVLTAKGMALFGEGKAEFTGRHDAHLNGKVIGTLKNSLFFSAQPFEITASTNNEGN
LKVRFPLRLTGKIDFLNNYALFLSPSAQQASWQVSARFNQYKYNQNFSAGNNENIMEA
HVGINGEANLDFLNIPLTIPEMRLPYTIITTPPLKDFSLWEKTGLKEFLKTTKQSFDLSVKA
QYKKNKHRHSITNPLAVLCEFISQSIKSFDRHFEKNRNNALDFVTKSYNETKIKFDKYKA
EKSHDELPRTFQIPGYTVPVVNVEVSPFTIEMSAFGYVFPKAVSMPSFSILGSDVRVPSYT
LILPSLELPVLHVPRNLKLSLPDFKELCTISHIFIPAMGNITYDFSFKSSVITLNTNAELFNQ
SDIVAHLLSSSSSVIDALQYKLEGTTRLTRKRGLKLATALSLSNKFVEGSHNSTVSLTTK
NMEVSVATTTKAQIPILRMNFKQELNGNTKSKPTVSSSMEFKYDFNSSMLYSTAKGAVD
HKLSLESLTSYFSIESSTKGDVKGSVLSREYSGTIASEANTYLNSKSTRSSVKLQGTSKID
DIWNLEVKENFAGEATLQRIYSLWEHSTKNHLQLEGLFFTNGEHTSKATLELSPWQMSA
LVQVHASQPSSFHDFPDLGQEVALNANTKNQKIRWKNEVRIHSGSFQSQVELSNDQEKA
HLDIAGSLEGHLRFLKNIILPVYDKSLWDFLKLDVTTSIGRRQHLRVSTAFVYTKNPNGY
SFSIPVKVLADKFIIPGLKLNDLNSVLVMPTFHVPFTDLQVPSCKLDFREIQIYKKLRTSSF
ALNLPTLPEVKFPEVDVLTKYSQPEDSLIPFFEITVPESQLTVSQFTLPKSVSDGIAALDLN
AVANKIADFELPTIIVPEQTIEIPSIKFSVPAGIVIPSFQALTARFEVDSPVYNATWSASLKN
KADYVETVLDSTCSSTVQFLEYELNVLGTHKIEDGTLASKTKGTFAHRDFSAEYEEDGK
YEGLQEWEGKAHLNIKSPAFTDLHLRYQKDKKGISTSAASPAVGTVGMDMDEDDDFSK
WNFYYSPQSSPDKKLTIFKTELRVRESDEETQIKVNWEEEAASGLLTSLKDNVPKATGV
LYDYVNKYHWEHTGLTLREVSSKLRRNLQNNAEWVYQGAIRQIDDIDVRFQKAASGTT
GTYQEWKDKAQNLYQELLTQEGQASFQGLKDNVFDGLVRVTQEFHMKVKHLIDSLIDF
LNFPRFQFPGKPGIYTREELCTMFIREVGTVLSQVYSKVHNGSEILFSYFQDLVITLPFELR
KHKLIDVISMYRELLKDLSKEAQEVFKAIQSLKTTEVLRNLQDLLQFIFQLIEDNIKQLKE
MKFTYLINYIQDEINTIFSDYIPYVFKLLKENLCLNLHKFNEFIQNELQEASQELQQIHQYI
MALREEYFDPSIVGWTVKYYELEEKIVSLIKNLLVALKDFHSEYIVSASNFTSQLSSQVE
QFLHRNIQEYLSILTDPDGKGKEKIAELSATAQEIIKSQAIATKKIISDYHQQFRYKLQDFS
DQLSDYYEKFIAESKRLIDLSIQNYHTFLIYITELLKKLQSTTVMNPYMKLAPGELTIIL
SEQ ID NO: 2 ApoB18 amino acid sequence
MDPPRPALLALLALPALLLLLLAGARAEEEMLENVSLVCPKDATRFKHLRKYTYNYEA
ESSSGVPGTADSRSATRINCKVELEVPQLCSFILKTSQCTLKEVYGFNPEGKALLKKTKN
SEEFAAAMSRYELKLAIPEGKQVFLYPEKDEPTYILNIKRGIISALLVPPETEEAKQVLFLD
TVYGNCSTHFTVKTRKGNVATEISTERDLGQCDRFKPIRTGISPLALIKGMTRPLSTLISSS
QSCQYTLDAKRKHVAEAICKEQHLFLPFSYKNKYGMVAQVTQTLKLEDTPKINSRFFGE
GTKKMGLAFESTKSTSPPKQAEAVLKTLQELKKLTISEQNIQRANLFNKLVTELRGLSDE
AVTSLLPQLIEVSSPITLQALVQCGQPQCSTHILQWLKRVHANPLLIDVVTYLVALIPEPS
AQQLREIFNMARDQRSRATLYALSHAVNNYHKTNPTGTQELLDIANYLMEQIQDDCTG
DEDYTYLILRVIGNMGQTMEQLTPELKSSILKCVQSTKPSLMIQKAAIQALRKMEPKDK
DQEVLLQTFLDDASPGDKRLAAYLMLMRSPSQADINKIVQILPWEQNEQVKNFVASHIA
NILNSEELDIQDLKKLVKEALKESQLPTVMDFRKFSRNYQLYKSVSLPSLDPASAKIEGN
LIFDPNNYLPKESMLKTTLTAFGFASADLIEIGLEGKGFEPTLEALFGKQGFFPDSVNKAL
YWVNGQVPDGVSKVLVDHFGYTKDDKHEQDMVNGIMLSVEKLIKDLKSKEVPEARAY
LRILGEELGFASLHDLQLLGKLLLMGARTLQGIPQMIGEV
SEQ ID NO: 3 ApoB15 amino acid sequence
MDPPRPALLALLALPALLLLLLAGARAEEEMLENVSLVCPKDATRFKHLRKYTYNYEA
ESSSGVPGTADSRSATRINCKVELEVPQLCSFILKTSQCTLKEVYGENPEGKALLKKTKN
SEEFAAAMSRYELKLAIPEGKQVFLYPEKDEPTYILNIKRGIISALLVPPETEEAKQVLFLD
TVYGNCSTHFTVKTRKGNVATEISTERDLGQCDRFKPIRTGISPLALIKGMTRPLSTLISSS
QSCQYTLDAKRKHVAEAICKEQHLFLPFSYKNKYGMVAQVTQTLKLEDTPKINSRFFGE
GTKKMGLAFESTKSTSPPKQAEAVLKTLQELKKLTISEQNIQRANLFNKLVTELRGLSDE
AVTSLLPQLIEVSSPITLQALVQCGQPQCSTHILQWLKRVHANPLLIDVVTYLVALIPEPS
AQQLREIFNMARDQRSRATLYALSHAVNNYHKTNPTGTQELLDIANYLMEQIQDDCTG
DEDYTYLILRVIGNMGQTMEQLTPELKSSILKCVQSTKPSLMIQKAAIQALRKMEPKDK
DQEVLLQTFLDDASPGDKRLAAYLMLMRSPSQADINKIVQILPWEQNEQVKNFVASHIA
NILNSEELDIQDLKKLVKEALKESQLPTVMDFRKFSRNYQLYKSVSLPSLDPASAKIEGN
LIFDPNNYLPKESMLKTTLTAFGFAS
SEQ ID NO: 4 ApoB12 amino acid sequence
MDPPRPALLALLALPALLLLLLAGARAEEEMLENVSLVCPKDATRFKHLRKYTYNYEA
ESSSGVPGTADSRSATRINCKVELEVPQLCSFILKTSQCTLKEVYGFNPEGKALLKKTKN
SEEFAAAMSRYELKLAIPEGKQVFLYPEKDEPTYILNIKRGIISALLVPPETEEAKQVLFLD
TVYGNCSTHFTVKTRKGNVATEISTERDLGQCDRFKPIRTGISPLALIKGMTRPLSTLISSS
QSCQYTLDAKRKHVAEAICKEQHLFLPFSYKNKYGMVAQVTQTLKLEDTPKINSRFFGE
GTKKMGLAFESTKSTSPPKQAEAVLKTLQELKKLTISEQNIQRANLFNKLVTELRGLSDE
AVTSLLPQLIEVSSPITLQALVQCGQPQCSTHILQWLKRVHANPLLIDVVTYLVALIPEPS
AQQLREIFNMARDQRSRATLYALSHAVNNYHKTNPTGTQELLDIANYLMEQIQDDCTG
DEDYTYLILRVIGNMGQTMEQLTPELKSSILKCVQSTKPSLMIQKAAIQALRKMEPKDK
DQEVL
SEQ ID NO: 5 ApoB8 to ApoB 12 amino acid sequence
VTSLLPQLIEVSSPITLQALVQCGQPQCSTHILQWLKRVHANPLLIDVVTYLVALIPEPSA
QQLREIFNMARDQRSRATLYALSHAVNNYHKTNPTGTQELLDIANYLMEQIQDDCTGD
EDYTYLILRVIGNMGQTMEQLTPELKSSILKCVQSTKPSLMIQKAAIQALRKMEPKDKD
QEVL
SEQ ID NO: 6 ApoB8 to ApoB15 amino acid sequence
VTSLLPQLIEVSSPITLQALVQCGQPQCSTHILQWLKRVHANPLLIDVVTYLVALIPEPSA
QQLREIFNMARDQRSRATLYALSHAVNNYHKTNPTGTQELLDIANYLMEQIQDDCTGD
EDYTYLILRVIGNMGQTMEQLTPELKSSILKCVQSTKPSLMIQKAAIQALRKMEPKDKD
QEVLLQTFLDDASPGDKRLAAYLMLMRSPSQADINKIVQILPWEQNEQVKNFVASHIAN
ILNSEELDIQDLKKLVKEALKESQLPTVMDFRKFSRNYQLYKSVSLPSLDPASAKIEGNLI
FDPNNYLPKESMLKTTLTAFGFAS
SEQ ID NO: 7 ApoB12 to ApoB 15 amino acid sequence
LLQTFLDDASPGDKRLAAYLMLMRSPSQADINKIVQILPWEQNEQVKNFVASHIANILNS
EELDIQDLKKLVKEALKESQLPTVMDFRKFSRNYQLYKSVSLPSLDPASAKIEGNLIFDP
NNYLPKESMLKTTLTAFGFAS

1. An isolated polypeptide which comprises an N-terminal portion of Apolipoprotein B100 (ApoB100), or a functional derivative thereof, wherein said polypeptide does not comprise any ApoB100 sequences beyond amino acid residue 816 of ApoB100.

2. The polypeptide of claim 1, wherein the ApoB100 has the amino acid sequence of SEQ ID NO: 1.

3. The polypeptide of claim 1, wherein said polypeptide is capable of inhibiting scavenger receptor-B1 (SR-B1) and/or activin receptor-like kinase 1 (ALK1).

4. The polypeptide of claim 1, wherein the polypeptide does not comprise amino acid residue 816 of ApoB100.

5. The polypeptide of claim 1, wherein the polypeptide comprises amino acid residues 363-544 of ApoB100, or a derivative thereof.

6. The polypeptide of claim 5, wherein the polypeptide comprises the amino acid sequence of SEQ ID NO: 5, or a derivative thereof.

7. The polypeptide of claim 6, wherein the polypeptide consists of the amino acid sequence of SEQ ID NO: 5, or a derivative thereof.

8. The polypeptide of claim 1, wherein the polypeptide comprises amino acid residues 1-544 of ApoB100, or a derivative thereof.

9. The polypeptide of claim 8, wherein the polypeptide comprises the amino acid sequence of SEQ ID NO: 4, or a derivative thereof.

10. The polypeptide of claim 9, wherein the polypeptide consists of the amino acid sequence of SEQ ID NO: 4, or a derivative thereof.

11. The polypeptide of claim 1, wherein the polypeptide comprises amino acid residues 544-684 of ApoB100, or a derivative thereof.

12. The polypeptide of claim 11, wherein the polypeptide comprises the amino acid sequence of SEQ ID NO: 7, or a derivative thereof.

13. The polypeptide of claim 12, wherein the polypeptide consists of the amino acid sequence of SEQ ID NO: 7, or a derivative thereof.

14. The polypeptide of claim 1, wherein the polypeptide comprises amino acid residues 363-684 of ApoB100, or a derivative thereof.

15. The polypeptide of claim 14, wherein the polypeptide comprises the amino acid sequence of SEQ ID NO: 6, or a derivative thereof.

16. The polypeptide of claim 15, wherein the polypeptide consists of the amino acid sequence of SEQ ID NO: 6, or a derivative thereof.

17. The polypeptide of claim 1, wherein the polypeptide comprises amino acid residues 1-684 of ApoB100, or a derivative thereof.

18. The polypeptide of claim 17, wherein the polypeptide comprises the amino acid sequence of SEQ ID NO: 3, or a derivative thereof.

19. The polypeptide of claim 18, wherein the polypeptide consists of the amino acid sequence of SEQ ID NO: 3, or a derivative thereof.

20. A polynucleotide comprising a nucleotide sequence encoding the polypeptide of claim 1.

21-24. (canceled)

25. A recombinant vector comprising the polynucleotide of claim 20.

26-32. (canceled)

33. A pharmaceutical composition comprising (i) the polypeptide of claim 1, (ii) a polynucleotide comprising a nucleotide sequence encoding said polypeptide, or (iii) a recombinant vector comprising said polynucleotide, and a pharmaceutically acceptable carrier or adjuvant.

34. A method of blocking uptake or transcytosis of low-density lipoprotein (LDL), very-low-density lipoprotein (VLDL), chylomicrons, and/or lipoprotein (a) (Lp(a)) by an endothelial cell, comprising contacting the endothelial cell with an effective amount of an inhibitor of scavenger receptor-B1 (SR-B1) and/or activin receptor-like kinase 1 (ALK1).

35-37. (canceled)

38. A method of treating or preventing atherosclerosis and/or an associated disease, disorder, or condition thereof in a subject in need thereof, comprising administering to the subject a therapeutically effective amount of an inhibitor of scavenger receptor-B1 (SR-B1) and/or activin receptor-like kinase 1 (ALK1).

39-49. (canceled)


SEQUENCE LISTING (7 sequences)

SEQ ID NO: 1 - protein, 4563 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEVIRKGSKNDFFLHYIFMENAFELPTGAGLQLQISSSGVIAPGAKAGVKLEVANMQAELVAKPSVSVEFVTNMGIIIPDFARSGVQMNTNFFHESGLEAHVALKAGKLKFIIPSPKRPVKLLSGGNTLHLVSTTKTEVIPPLIENRQSWSVCKQVFPGLNYCTSGAYSNASSTDSASYYPLTGDTRLELELRPTGEIEQYSVSATYELQREDRALVDTLKFVTQAEGAKQTEATMTFKYNRQSMTLSSEVQIPDFDVDLGTILRVNDESTEGKTSYRLTLDIQNKKITEVALMGHLSCDTKEERKIKGVISIPRLQAEARSEILAHWSPAKLLLQMDSSATAYGSTVSKRVAWHYDEEKIEFEWNTGTNVDTKKMTSNFPVDLSDYPKSLHMYANRLLDHRVPQTDMTFRHVGSKLIVAMSSWLQKASGSLPYTQTLQDHLNSLKEFNLQNMGLPDFHIPENLFLKSDGRVKYTLNKNSLKIEIPLPFGGKSSRDLKMLETVRTPALHFKSVGFHLPSREFQVPTFTIPKLYQLQVPLLGVLDLSTNVYSNLYNWSASYSGGNTSTDHFSLRARYHMKADSVVDLLSYNVQGSGETTYDHKNTFTLSYDGSLRHKFLDSNIKFSHVEKLGNNPVSKGLLIFDASSSWGPQMSASVHLDSKKKQHLFVKEVKIDGQFRVSSFYAKGTYGLSCQRDPNTGRLNGESNLRFNSSYLQGTNQITGRYEDGTLSLTSTSDLQSGIIKNTASLKYENYELTLKSDTNGKYKNFATSNKMDMTFSKQNALLRSEYQADYESLRFFSLLSGSLNSHGLELNADILGTDKINSGAHKATLRIGQDGISTSATTNLKCSLLVLENELNAELGLSGASMKLTTNGRFREHNAKFSLDGKAALTELSLGSAYQAMILGVDSKNIFNFKVSQEGLKLSNDMMGSYAEMKFDHTNSLNIAGLSLDFSSKLDNIYSSDKFYKQTVNLQLQPYSLVTTLNSDLKYNALDLTNNGKLRLEPLKLHVAGNLKGAYQNNEIKHIYAISSAALSASYKADTVAKVQGVEFSHRLNTDIAGLASAIDMSTNYNSDSLHFSNVFRSVMAPFTMTIDAHTNGNGKLALWGEHTGQLYSKFLLKAEPLAFTFSHDYKGSTSHHLVSRKSISAALEHKVSALLTPAEQTGTWKLKTQFNNNEYSQDLDAYNTKDKIGVELTGRTLADLTLLDSPIKVPLLLSEPINIIDALEMRDAVEKPQEFTIVAFVKYDKNQDVHSINLPFFETLQEYFERNRQTIIVVLENVQRNLKHINIDQFVRKYRAALGKLPQQANDYLNSFNWERQVSHAKEKLTALTKKYRITENDIQIALDDAKINFNEKLSQLQTYMIQFDQYIKDSYDLHDLKIAIANIIDEIIEKLKSLDEHYHIRVNLVKTIHDLHLFIENIDFNKSGSSTASWIQNVDTKYQIRIQIQEKLQQLKRHIQNIDIQHLAGKLKQHIEAIDVRVLLDQLGTTISFERINDILEHVKHFVINLIGDFEVAEKINAFRAKVHELIERYEVDQQIQVLMDKLVELAHQYKLKETIQKLSNVLQQVKIKDYFEKLVGFIDDAVKKLNELSFKTFIEDVNKFLDMLIKKLKSFDYHQFVDETNDKIREVTQRLNGEIQALELPQKAEALKLFLEETKATVAVYLESLQDTKITLIINWLQEALSSASLAHMKAKFRETLEDTRDRMYQMDIQQELQRYLSLVGQVYSTLVTYISDWWTLAAKNLTDFAEQYSIQDWAKRMKALVEQGFTVPEIKTILGTMPAFEVSLQALQKATFQTPDFIVPLTDLRIPSVQINFKDLKNIKIPSRFSTPEFTILNTFHIPSFTIDFVEMKVKIIRTIDQMLNSELQWPVPDIYLRDLKVEDIPLARITLPDFRLPEIAIPEFIIPTLNLNDFQVPDLHIPEFQLPHISHTIEVPTFGKLYSILKIQSPLFTLDANADIGNGTTSANEAGIAASITAKGESKLEVLNFDFQANAQLSNPKINPLALKESVKFSSKYLRTEHGSEMLFFGNAIEGKSNTVASLHTEKNTLELSNGVIVKINNQLTLDSNTKYFHKLNIPKLDFSSQADLRNEIKTLLKAGHIAWTSSGKGSWKWACPRFSDEGTHESQISFTIEGPLTSFGLSNKINSKHLRVNQNLVYESGSLNFSKLEIQSQVDSQHVGHSVLTAKGMALFGEGKAEFTGRHDAHLNGKVIGTLKNSLFFSAQPFEITASTNNEGNLKVRFPLRLTGKIDFLNNYALFLSPSAQQASWQVSARFNQYKYNQNFSAGNNENIMEAHVGINGEANLDFLNIPLTIPEMRLPYTIITTPPLKDFSLWEKTGLKEFLKTTKQSFDLSVKAQYKKNKHRHSITNPLAVLCEFISQSIKSFDRHFEKNRNNALDFVTKSYNETKIKFDKYKAEKSHDELPRTFQIPGYTVPVVNVEVSPFTIEMSAFGYVFPKAVSMPSFSILGSDVRVPSYTLILPSLELPVLHVPRNLKLSLPDFKELCTISHIFIPAMGNITYDFSFKSSVITLNTNAELFNQSDIVAHLLSSSSSVIDALQYKLEGTTRLTRKRGLKLATALSLSNKFVEGSHNSTVSLTTKNMEVSVATTTKAQIPILRMNFKQELNGNTKSKPTVSSSMEFKYDFNSSMLYSTAKGAVDHKLSLESLTSYFSIESSTKGDVKGSVLSREYSGTIASEANTYLNSKSTRSSVKLQGTSKIDDIWNLEVKENFAGEATLQRIYSLWEHSTKNHLQLEGLFFTNGEHTSKATLELSPWQMSALVQVHASQPSSFHDFPDLGQEVALNANTKNQKIRWKNEVRIHSGSFQSQVELSNDQEKAHLDIAGSLEGHLRFLKNIILPVYDKSLWDFLKLDVTTSIGRRQHLRVSTAFVYTKNPNGYSFSIPVKVLADKFIIPGLKLNDLNSVLVMPTFHVPFTDLQVPSCKLDFREIQIYKKLRTSSFALNLPTLPEVKFPEVDVLTKYSQPEDSLIPFFEITVPESQLTVSQFTLPKSVSDGIAALDLNAVANKIADFELPTIIVPEQTIEIPSIKFSVPAGIVIPSFQALTARFEVDSPVYNATWSASLKNKADYVETVLDSTCSSTVQFLEYELNVLGTHKIEDGTLASKTKGTFAHRDFSAEYEEDGKYEGLQEWEGKAHLNIKSPAFTDLHLRYQKDKKGISTSAASPAVGTVGMDMDEDDDFSKWNFYYSPQSSPDKKLTIFKTELRVRESDEETQIKVNWEEEAASGLLTSLKDNVPKATGVLYDYVNKYHWEHTGLTLREVSSKLRRNLQNNAEWVYQGAIRQIDDIDVRFQKAASGTTGTYQEWKDKAQNLYQELLTQEGQASFQGLKDNVFDGLVRVTQEFHMKVKHLIDSLIDFLNFPRFQFPGKPGIYTREELCTMFIREVGTVLSQVYSKVHNGSEILFSYFQDLVITLPFELRKHKLIDVISMYRELLKDLSKEAQEVFKAIQSLKTTEVLRNLQDLLQFIFQLIEDNIKQLKEMKFTYLINYIQDEINTIFSDYIPYVFKLLKENLCLNLHKFNEFIQNELQEASQELQQIHQYIMALREEYFDPSIVGWTVKYYELEEKIVSLIKNLLVALKDFHSEYIVSASNFTSQLSSQVEQFLHRNIQEYLSILTDPDGKGKEKIAELSATAQEIIKSQAIATKKIISDYHQQFRYKLQDFSDQLSDYYEKFIAESKRLIDLSIQNYHTFLIYITELLKKLQSTTVMNPYMKLAPGELTIIL

SEQ ID NO: 2 - protein, 816 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFASADLIEIGLEGKGFEPTLEALFGKQGFFPDSVNKALYWVNGQVPDGVSKVLVDHFGYTKDDKHEQDMVNGIMLSVEKLIKDLKSKEVPEARAYLRILGEELGFASLHDLQLLGKLLLMGARTLQGIPQMIGEV

SEQ ID NO: 3 - protein, 684 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFAS

SEQ ID NO: 4 - protein, 544 aa, Homo sapiens
MDPPRPALLALLALPALLLLLLAGARAEEEMLENVSLVCPKDATRFKHLRKYTYNYEAESSSGVPGTADSRSATRINCKVELEVPQLCSFILKTSQCTLKEVYGFNPEGKALLKKTKNSEEFAAAMSRYELKLAIPEGKQVFLYPEKDEPTYILNIKRGIISALLVPPETEEAKQVLFLDTVYGNCSTHFTVKTRKGNVATEISTERDLGQCDRFKPIRTGISPLALIKGMTRPLSTLISSSQSCQYTLDAKRKHVAEAICKEQHLFLPFSYKNKYGMVAQVTQTLKLEDTPKINSRFFGEGTKKMGLAFESTKSTSPPKQAEAVLKTLQELKKLTISEQNIQRANLFNKLVTELRGLSDEAVTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVL

SEQ ID NO: 5 - protein, 182 aa, Homo sapiens
VTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVL

SEQ ID NO: 6 - protein, 322 aa, Homo sapiens
VTSLLPQLIEVSSPITLQALVQCGQPQCSTHILQWLKRVHANPLLIDVVTYLVALIPEPSAQQLREIFNMARDQRSRATLYALSHAVNNYHKTNPTGTQELLDIANYLMEQIQDDCTGDEDYTYLILRVIGNMGQTMEQLTPELKSSILKCVQSTKPSLMIQKAAIQALRKMEPKDKDQEVLLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFAS

SEQ ID NO: 7 - protein, 141 aa, Homo sapiens
LLQTFLDDASPGDKRLAAYLMLMRSPSQADINKIVQILPWEQNEQVKNFVASHIANILNSEELDIQDLKKLVKEALKESQLPTVMDFRKFSRNYQLYKSVSLPSLDPASAKIEGNLIFDPNNYLPKESMLKTTLTAFGFAS